(19) United States
(12) Patent Application Publication
(10) Pub. No.: US 2023/0346590 A1
(43) Pub. Date: Nov. 2, 2023

(54) MECHANICAL TUBAL OCCLUSION CONTRACEPTIVE DEVICE

(71) Applicant: ECLIPSE MEDICAL, INC., Roseville, MN (US)

(72) Inventors: Gladwin Das, Arden Hills, MN (US); Dilip Das, Arden Hills, MN (US)

(21) Appl. No.: 18/247,674

(22) PCT Filed: Oct. 11, 2021
(86) PCT No.: PCT/US2021/054441

§ 371 Date: Apr. 3, 2023

Related U.S. Application Data
(60) Provisional application No. 63/090,945, filed on Oct. 13, 2020.

(51) Int. Cl. A61F 6/22

(52) U.S. Cl. CPC A61F 6/22

(57) ABSTRACT

A tubal occlusion contraceptive device/appliance (100/200) and methods of implant and removal. The device (100) includes a plurality of barbs (124) with some preferentially resisting movement in a first axial direction and others preferentially resisting movement in a second opposite axial direction. The device (100) is implanted in a Fallopian Tube (FT) through a sheath. The appliance (200) includes an oversized cap (220) to prevent continued insertion of appliance (200) into a Fallopian Tube (FT), and a plurality of barbs (240) to facilitate a first axial direction slide fit insertion of the appliance (200) into a Fallopian Tube (FT) while preferentially resisting axial movement in a second opposite axial direction out from the Fallopian Tube (FT) once inserted. The barbs (240) can be replaced with screw threads (250).

### BACKGROUND

The ability to use minimally invasive techniques to permanently occlude fallopian tubes has been unsuccessful for a variety of reasons, the most important being that simple plugs and fallopian tubular occluders have not been designed to primarily occlude the fallopian tube. They have been designed for ease of deployment and for hysteroscopic delivery that can be performed in office based practices. This leads to suboptimal results.

Accordingly, a need exists for a highly effective yet minimally invasive mechanical tubal occlusion contraceptive device.

### SUMMARY OF THE INVENTION

A first aspect of the invention is a mechanical contraceptive device/appliance.

A first embodiment of the first aspect of the invention is a mechanical tubal occlusion contraceptive device that includes an axially elongated scar-inducing occlusion element and a pair of axially elongated anchors. The occlusion element has first and second axial end portions terminating at first and second axial ends, respectively. The pair of anchors include a leading anchor and a trailing anchor. The leading anchor is secured to and extends in a first axial direction from the first axial end portion of the occlusion element while the trailing anchor is secured to and extends in a second axial direction opposite the first axial direction from the second axial end of the occlusion element. The leading anchor includes a plurality of radially extending barbs operable for resisting axial movement of the leading anchor relative to a fallopian tube into which the mechanical tubal occlusion contraceptive device has been implanted. The trailing anchor includes a plurality of radially extending barbs operable for resisting axial movement of the trailing anchor relative to a fallopian tube into which the mechanical tubal occlusion contraceptive device has been implanted, and a head proximate a free axial end of the trailing anchor, configured and arranged for releasable engagement by a pusher delivery catheter for delivery of the mechanical tubal occlusion contraceptive device into a fallopian tube.

A second embodiment of the first aspect of the invention is a mechanical tubal occlusion contraceptive device that includes an axially elongated shaft, a head and a plurality of barbs. The shaft has a proximal axial end spaced in a first axial direction from a distal tip, and defines a second axial direction opposite the first axial direction. The head projects in the first axial direction from the proximal end of the shaft, and is configured and arranged for releasable engagement by a pusher delivery catheter for delivery of the mechanical tubal occlusion contraceptive device into a fallopian tube. The plurality of barbs extend radially from the shaft intermediate the head and the distal tip of the shaft, and collectively resist axial movement of the mechanical tubal occlusion contraceptive device in both the first and second axial directions relative to a fallopian tube into which the mechanical tubal occlusion contraceptive device has been implanted.

A third embodiment of the first aspect of the invention is a tubal occlusion contraceptive appliance that includes an axially elongated shaft, a cap and a plurality of barbs. The shaft has a proximal axial end spaced in a first axial direction from a distal axial end, and defines a second axial direction opposite the first axial direction. A guideway extends through the shaft proximate the distal axial end of the shaft for accommodating passage of a guidewire for guiding insertion of the appliance into a fallopian tube. The cap is positioned proximate the proximal axial end of the shaft, and is sized, configured and arranged to inhibit axial travel of the cap into a fallopian tube. The barbs extend radially from the shaft intermediate the distal axial end of the shaft and the cap, and are configured and arranged to facilitate slide fit insertion of the shaft into a fallopian tube in the second axial direction while preferentially resisting axial movement of the shaft in the first axial direction out from the fallopian tube once inserted.

A fourth embodiment of the first aspect of the invention is a tubal occlusion contraceptive appliance that includes an axially elongated shaft, a cap, a pair of axially extending guideways, and a plurality of barbs. The shaft has a proximal axial end spaced in a first axial direction from a distal tip, and defines a second axial direction opposite the first axial direction. The cap is positioned proximate the proximal end of the shaft, and is sized, configured and arranged to inhibit axial travel of the cap into a fallopian tube. The pair of guideways includes a proximal guideway and a distal guideway, both operable for accommodating passage of a guidewire for guiding insertion of the appliance into a fallopian tube. The proximal guideway extends through the cap in radially spaced relationship from the shaft. The distal guideway extends through the shaft proximate the distal tip of the shaft. The barbs extend radially from the shaft intermediate the distal axial end of the shaft and the cap, and are configured and arranged to facilitate slide fit insertion of the shaft into a fallopian tube in the second axial direction while preferentially resisting axial movement of the shaft in the first axial direction out from the fallopian tube once inserted.

A fifth embodiment of the first aspect of the invention is a tubal occlusion contraceptive appliance that includes an axially elongated shaft, a cap, a pair of axially extending guideways, and screw threads. The shaft has a proximal axial end spaced in a first axial direction from a distal tip, and defines a second axial direction opposite the first axial direction. The cap is positioned proximate the proximal end of the shaft, and is sized, configured and arranged to inhibit axial travel of the cap into a fallopian tube. The pair of guideways includes a proximal guideway and a distal guideway, both operable for accommodating passage of a guidewire for guiding insertion of the appliance into a fallopian tube. The proximal guideway extends through the cap in radially spaced relationship from the shaft. The distal guideway extends through the shaft proximate the distal tip of the shaft. The screw threads extend radially around the shaft intermediate the distal tip of the shaft and the cap, and are configured and arranged to (i) facilitate slide fit insertion of the shaft into a fallopian tube in the second axial direction with or without rotation of the appliance in a first direction while preferentially resisting sliding axial movement of the shaft in the first axial direction out from the fallopian tube once inserted, and (ii) effect withdrawal of the tubal occlusion contraceptive appliance from the fallopian tube upon rotation of the appliance in a second direction opposite the first direction.

A sixth embodiment of the first aspect of the invention is a tubal occlusion contraceptive appliance that includes an axially elongated shaft, a cap, a pair of axially extending guideways, and screw threads. The shaft has a proximal axial end spaced in a first axial direction from a distal tip, and defines a second axial direction opposite the first axial direction. The cap is positioned proximate the proximal end of the shaft, and is sized, configured and arranged to inhibit axial travel of the cap into a fallopian tube. The pair of guideways includes a proximal guideway and a distal guideway, both operable for accommodating passage of a guidewire for guiding insertion of the appliance into a fallopian tube. The proximal guideway extends through the cap in radially spaced relationship from the shaft. The distal guideway extends through the shaft proximate the distal tip of the shaft. The screw threads extend radially around the shaft intermediate the distal tip of the shaft and the cap for effecting axial insertion of the tubal occlusion contraceptive appliance into a fallopian tube upon rotation of the appliance in a first direction, and withdrawal of the tubal occlusion contraceptive appliance from the fallopian tube upon rotation of the appliance in a second direction opposite the first direction.

A second aspect of the invention is a method of implanting the mechanical contraceptive devices/appliances in accordance with the first aspect of the invention.

A first embodiment of the second aspect of the invention includes the steps of (i) transvaginal introduction of a sheath having a lumen into a fallopian tube with an introduction end of the sheath exterior the vagina and a delivery end of the sheath within the fallopian tube, (ii) axially pushing the mechanical tubal occlusion contraceptive device in accordance with the first embodiment of the first aspect of the invention along the lumen of the sheath with a delivery device from the introduction end to proximate the delivery end of the sheath, and (iii) withdrawing the delivery device and the sheath while leaving the mechanical tubal occlusion contraceptive device within the fallopian tube.

A second embodiment of the second aspect of the invention includes the steps of (i) transvaginal introduction of a sheath having a lumen into a fallopian tube with an introduction end of the sheath exterior the vagina and a delivery end of the sheath within the fallopian tube, (ii) axially pushing the mechanical tubal occlusion contraceptive device in accordance with the second embodiment of the first aspect of the invention along the lumen of the sheath with a delivery device from the introduction end to proximate the delivery end of the sheath, and (iii) withdrawing the delivery device and the sheath while leaving the mechanical tubal occlusion contraceptive device within the fallopian tube.

A third embodiment of the second aspect of the invention includes the steps of (i) transvaginal introduction of a guidewire into a fallopian tube with a first end of the guidewire exterior the vagina and a second end of the guidewire within the fallopian tube, (ii) sliding the first end of the guidewire through the guideway in a tubal occlusion contraceptive appliance in accordance with the third embodiment of the first aspect of the invention, (iii) axially pushing the tubal occlusion contraceptive appliance along the guidewire towards the second end of the guidewire with a delivery device until the shaft is positioned within the fallopian tube and the cap is seated over the opening from the uterus to the fallopian tube, and (iv) withdrawing the delivery device and the guidewire while leaving the tubal occlusion contraceptive appliance extending into the fallopian tube.

A fourth embodiment of the second aspect of the invention includes the steps of (i) transvaginal introduction of a guidewire into a fallopian tube with a first end of the guidewire exterior the vagina and a second end of the guidewire within the fallopian tube, (ii) serially sliding the first end of the guidewire through the distal guideway and then through the proximal guideway in a tubal occlusion contraceptive appliance in accordance with the fourth embodiment of the first aspect of the invention, (iii) axially pushing the tubal occlusion contraceptive appliance along the guidewire towards the second end of the guidewire with a delivery device until the shaft is positioned within the fallopian tube and the cap is seated over the opening from the uterus into the fallopian tube, and (iv) withdrawing the delivery device and the guidewire while leaving the tubal occlusion contraceptive appliance extending into the fallopian tube.

A fifth embodiment of the second aspect of the invention includes the steps of (i) transvaginal introduction of a guidewire into a fallopian tube with a first end of the guidewire exterior the vagina and a second end of the guidewire within the fallopian tube, (ii) serially sliding the first end of the guidewire through the distal guideway and then through the proximal guideway in a tubal occlusion contraceptive appliance in accordance with the fifth embodiment of the first aspect of the invention, (iii) axially pushing the tubal occlusion contraceptive appliance along the guidewire towards the second end of the guidewire with a delivery device until the shaft is positioned within the fallopian tube and the cap is seated over the opening from the uterus into the fallopian tube, and (iv) withdrawing the delivery device and the guidewire while leaving the tubal occlusion contraceptive appliance extending into the fallopian tube.

A sixth embodiment of the second aspect of the invention includes the steps of (i) transvaginal introduction of a guidewire into a fallopian tube with a first end of the guidewire exterior the vagina and a second end of the guidewire within the fallopian tube, (ii) serially sliding the first end of the guidewire through the distal guideway and then through the proximal guideway in a tubal occlusion contraceptive appliance in accordance with the sixth embodiment of the first aspect of the invention, (iii) axially pushing the tubal occlusion contraceptive appliance along the guidewire towards the second end of the guidewire with a delivery device until the screw threads on the shaft contact the fallopian tube, (iv) rotating the tubal occlusion contraceptive appliance in the first direction until the cap is seated over the opening from the uterus into the fallopian tube, and (v) withdrawing the delivery device and the guidewire while leaving the tubal occlusion contraceptive appliance extending into the fallopian tube.

A third aspect of the invention is a method of removing an implanted tubal occlusion contraceptive appliance.

A first embodiment of the third aspect of the invention includes the steps of (i) gripping the cap of a previously implanted tubal occlusion contraceptive appliance according to the fifth embodiment of the first aspect of the invention with a forceps, (ii) rotating the cap with the forceps about the axial axis of the implanted tubal occlusion contraceptive appliance whereby the screw threads on the implanted tubal occlusion contraceptive appliance effect axial travel of the implanted tubal occlusion contraceptive appliance completely out from the fallopian tube to create a withdrawn appliance, and then (iii) removing the withdrawn appliance completely from the body. Alternately the delivery cable is rotated clockwise and the appliance is torqued out because of the reverse threads. This is possible before the delivery cable is rotated counter clockwise to release it.

A second embodiment of the third aspect of the invention includes the steps of (i) gripping the cap of a previously implanted tubal occlusion contraceptive appliance according to the sixth embodiment of the first aspect of the invention with a forceps, (ii) rotating the cap with the forceps about the axial axis of the implanted tubal occlusion contraceptive appliance whereby the screw threads on the implanted tubal occlusion contraceptive appliance effect axial travel of the implanted tubal occlusion contraceptive appliance completely out from the fallopian tube to create a withdrawn appliance, and then (iii) removing the withdrawn appliance completely from the body. Alternately the delivery cable is rotated clockwise and the appliance is torqued out because of the reverse threads. This is possible before the delivery cable is rotated counter clockwise to release it.

### BRIEF DESCRIPTION OF THE DRAWINGS

FIG. 1 is a side view of an axially bent first embodiment of a mechanical tubal occlusion contraceptive device in accordance with the invention.

FIG. 2 is an exploded side view of the contraceptive device depicted in FIG. 1.

FIG. 3 is a perspective view of one of the anchors depicted in FIG. 1.

FIG. 4 is a side view of the anchor depicted in FIG. 3.

FIG. 5 is a schematic frontal plane view of the human female reproductive system.

FIG. 6 is the schematic frontal plane view of the human female reproductive system of FIG. 5 depicting transvaginal catheter introduction of a guidewire into a fallopian tube.

FIG. 7 is the schematic frontal plane view of the human female reproductive system of FIG. 6 after withdrawal of the catheter, leaving the transvaginal guidewire extending into the fallopian tube.

FIG. 8 is the schematic frontal plane view of the human female reproductive system of FIG. 7 depicting transvaginal introduction of an introducer sheath into the fallopian tube along the guidewire.

FIG. 9 is the schematic frontal plane view of the human female reproductive system of FIG. 8 after withdrawal of the guidewire, leaving the transvaginal introducer sheath extending into the fallopian tube.

FIG. 10 is a side view of the contraceptive device depicted in FIG. 1 tethered to the distal tip of a pusher delivery catheter.

FIG. 11 is the schematic frontal plane view of the human female reproductive system of FIG. 9 after introduction of the contraceptive device depicted in FIG. 10 into the fallopian tube through the introducer sheath via the pusher delivery catheter.

FIG. 12 is an enlarged side view of the introduced contraceptive device in FIG. 11.

FIG. 13 is the schematic frontal plane view of the human female reproductive system of FIG. 11 after withdrawal of the pusher delivery catheter and introducer sheath, leaving the contraceptive device depicted in FIG. 1 within the fallopian tube.

FIG. 14 is the schematic frontal plane view of the human female reproductive system of FIG. 13 after delivery of a contraceptive device depicted in FIG. 1 within both fallopian tubes.

FIG. 15 is the schematic frontal plane view of the human female reproductive system of FIG. 14 after growth of scar tissue around the occlusion element of each delivered contraceptive device.

FIG. 16 is a side perspective view of a second embodiment of a mechanical tubal occlusion contraceptive device in accordance with the invention.

FIG. 17 is a side perspective view of a third embodiment of a mechanical tubal occlusion contraceptive device in accordance with the invention.

FIG. 18 is a side view of the mechanical tubal occlusion contraceptive device depicted in FIG. 17.

FIG. 19 is a side view of the mechanical tubal occlusion contraceptive device depicted in FIG. 18 rotated 90° about the axial axis of the device.

FIG. 20 is a distal end view of the mechanical tubal occlusion contraceptive device depicted in FIG. 17.

FIG. 21 is a proximal end view of the mechanical tubal occlusion contraceptive device depicted in FIG. 17.

FIG. 22 is the schematic frontal plane view of the human female reproductive system of FIG. 5 after delivery of a contraceptive device depicted in FIG. 16 within both fallopian tubes.

FIG. 23 is a side view of a first embodiment of a tubal occlusion contraceptive appliance in accordance with the invention.

FIG. 24 is a side view of the first embodiment of the tubal occlusion contraceptive appliance depicted in FIG. 23, rotated 90° about the axial axis of the appliance.

FIG. 25 is the schematic frontal plane view of the human female reproductive system of FIG. 6 after withdrawal of the catheter, leaving the transvaginal guidewire extending into the fallopian tube.

FIG. 26 is the schematic frontal plane view of the human female reproductive system of FIG. 25 depicting delivery of the tubal occlusion contraceptive appliance depicted in FIG. 23 into the fallopian tube along the guidewire.

FIG. 27 is the schematic frontal plane view of the human female reproductive system of FIG. 26 after delivery of a contraceptive device as depicted in FIG. 23 within both fallopian tubes and removal of the guidewires.

FIG. 28 is a side view of a second embodiment of a tubal occlusion contraceptive appliance in accordance with the invention.

FIG. 29 is a side view of the tubal occlusion contraceptive appliance depicted in FIG. 28 rotated 90° about the axial axis of the appliance.

FIG. 30 is a distal end view of the tubal occlusion contraceptive appliance depicted in FIG. 28.

FIG. 31 is a proximal end view of the tubal occlusion contraceptive appliance depicted in FIG. 28.

FIG. 32 is a side view of a third embodiment of a tubal occlusion contraceptive appliance in accordance with the invention.

FIG. 33 is a side view of the tubal occlusion contraceptive appliance depicted in FIG. 32 slidably engaged upon a guidewire and threadably engaged with the distal tip of a pusher delivery catheter.

FIG. 34 is a perspective view of a fourth embodiment of a tubal occlusion contraceptive appliance in accordance with the invention.

FIG. 35 is a side view of the tubal occlusion contraceptive appliance depicted in FIG. 34.

FIG. 36 is a side view of the tubal occlusion contraceptive appliance depicted in FIG. 35 rotated 90° about the axial axis of the appliance.

FIG. 37 is a distal end view of the tubal occlusion contraceptive appliance depicted in FIG. 34.

FIG. 38 is a proximal end view of the tubal occlusion contraceptive appliance depicted in FIG. 34.

### DETAILED DESCRIPTION OF A PREFERRED EMBODIMENT

### Nomenclature

Nomenclature Table

| REF NO. | DESCRIPTION |
|---|---|
| 100 | Mechanical Tubal Occlusion Contraceptive Device |
| 110 | Occlusion Element |
| 110₁ | First Axial End Portion of Occlusion Element |
| 110₂ | Second Axial End Portion of Occlusion Element |
| 110₃ | Central Portion of Occlusion Element |
| 110ₓ₁ | First Axial End of Occlusion Element |
| 110ₓ₂ | Second Axial End of Occlusion Element |
| 120 | Anchor |
| 120¹ | Leading Anchor |
| 120² | Trailing Anchor |
| 120ₓ₁ | Free Axial End of Anchor |
| 120ₓ₂ | Secured Axial End of Anchor |
| 121 | Sleeve |
| 122 | Head |
| 123 | Shaft |
| 123ₓ₁ | Proximal Axial End of Shaft |
| 123ₓ₂ | Distal Axial Tip of Shaft |
| 124 | Barb |
| 124₁ | Barb Preferentially Resisting Movement in the |
| | First Axial Direction |
| 124₂ | Barb Preferentially Resisting Movement in the |
| | Second Axial Direction |
| 126 | Crimp |
| 127 | Radial Visual Inspection Hole Through Sleeve |
| 128 | Axial Orifice in Head |
| 129 | Radial Hole Through Head |
| 200 | Tubal Occlusion Contraceptive Appliance |
| 210 | Shaft |
| 210ₓ₁ | Proximal First Axial End of Shaft |
| 210ₓ₂ | Distal Second Axial End of Shaft (Distal Tip of Shaft) |
| 210s | Side of Shaft |
| 218 | Distal Guideway Through Shaft |
| 220 | Cap |
| 221 | Knurled Circumferential Surface |
| 227 | Internally Threaded Axial Orifice in Cap |
| 228 | Proximal Guideway Through Cap |
| 229 | Radial Hole Through Cap |
| 240 | Barb |
| 250 | Screw Threads |
| 251 | Screw Thread Segment |
| x | Axial Direction |
| x₁ | First Axial Direction |
| x₂ | Second Axial Direction |
| r | Radial Direction |
| PdC | Pusher Delivery Catheter |
| PdCₓ₁ | Distal Tip of Pusher Delivery Catheter |
| PdCₓ₂ | Proximal End of Pusher Delivery Catheter |
| H | Hub of Pusher Delivery Catheter |
| FT | Fallopian Tube |
| G | Guidewire |
| GdC | Guidewire Delivery Catheter |
| S | Vascular Sheath |
| T | Tether |

### Mechanical Tubal Occlusion Contraceptive Device 100

### First Embodiment

### Device

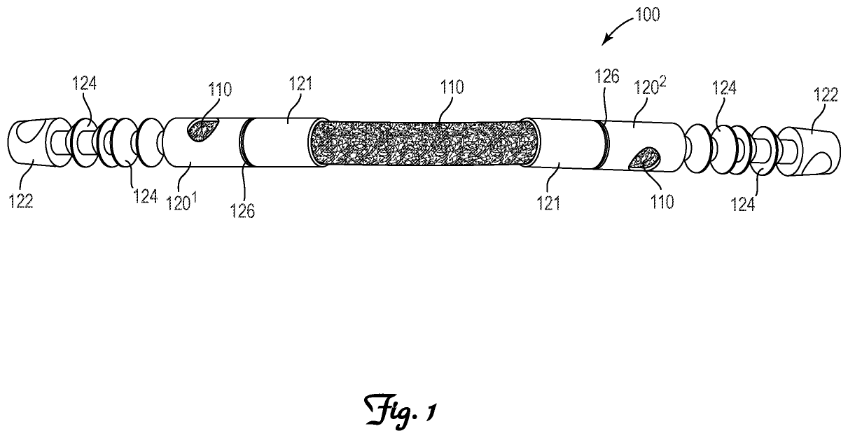

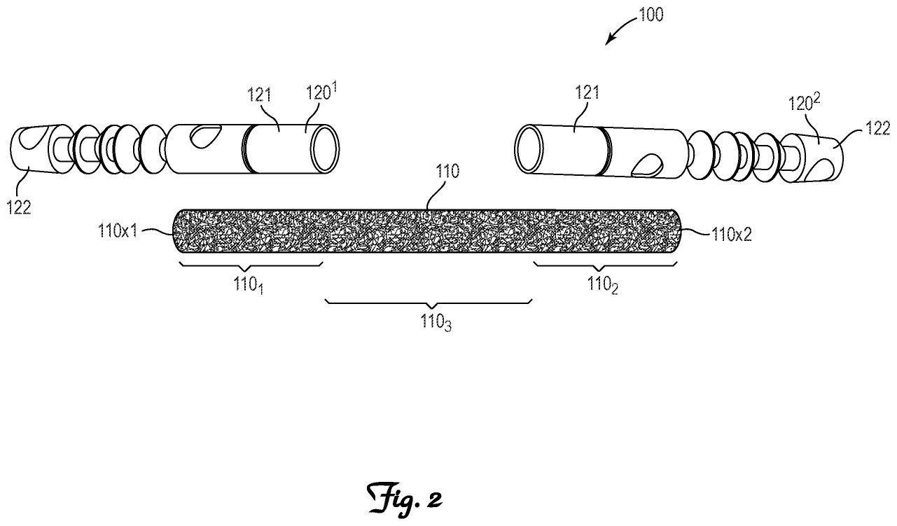

Referring to FIGS. 1 and 2, a first embodiment of a mechanical tubal occlusion contraceptive device 100 includes an axially x elongated scar-inducing occlusion element 110 and a pair of axially elongated anchors 120.

Polyester fabric and fibers have a long history dating back to the 1950s for its use in permanent implants for instance in surgical repair of cardiac septal defects and are biocompatible. They are also known to produce significant scar formation and do not biodegrade and hence provide permanent repair.

A thick polyester cord 110 positioned within the fallopian tubes FT for a few weeks would lead to scar formation and permanent occlusion of the fallopian tubes FT. However, due to the contractility of the fallopian tubes FT a simple cord 110 is likely to be expelled into the uterus or abdominal cavity.

The cord 110 is secured in place in the fallopian tubes FT by an anchor 120 at each axial end 110ₓ₁ and 110ₓ₂. After placement of the mechanical tubal occlusion contraceptive device 100 into a fallopian tube FT the mechanical tubal occlusion contraceptive device 100 will remain fixed in place and lead to scar formation in a matter of weeks leading to complete occlusion of the fallopian tubes FT. The anchors 120 can be CNC micro machined from biocompatible metallic alloys. In the preferred embodiment the anchors 120 are machined from 316 L stainless steel alloy that has been used in human implants for decades in orthopedic screws, plates etc. and is biocompatible, or from solid rods of polymers such as PEEK (Polyether Ether Ketone). The anchors 120 could potentially be manufactured from biocompatible polymers that biodegrade over time, for instance PLLA or PGA, whereby the anchors 120 biodegrade and no metallic components remain in the body once the cord 110 leads to scar formation and occlusion of the fallopian tubes FT.

The occlusion element 110 is conveniently divided into first and second axial end portions 110₁ and 110₂ and a central portion 110₃ therebetween. The first and second axial end portions 110₁ and 110₂ terminate at first and second axial ends 110ₓ₁ and 110ₓ₂, respectively. A preferred occlusion element 110 is a multifilament woven polyester cord around 1 mm in diameter and 30 to 50 mm in length.

The pair of anchors 120 include a leading anchor 120¹ and a trailing anchor 120². The leading anchor 120¹ is secured to and extends in a first axial direction x₁ from the first axial end portion 110₁ of the occlusion element 110 while the trailing anchor 120² is secured to and extends in a second axial direction x₂ opposite the first axial direction x₁ from the second axial end 110ₓ₂ of the occlusion element 110.

Both leading and trailing anchors 120¹ and 120² each preferably include an axially x extending hollow tube or sleeve 121 configured and arranged to encase the respective first and second axial end portions 110₁ and 110₂ of the occlusion element 110, thereby enabling a robust attachment of the leading and trailing anchors 120¹ and 120² to their respective first and second axial ends 110ₓ₁ and 110ₓ₂ of the occlusion element 110 such as by mechanical crimping of the sleeves 121 onto the occlusion element 110. A visual inspection hole 127 about 1 mm in diameter can be provided through the sleeve 121 at the lower end of the sleeve 121 for confirming complete insertion of the occlusion element 110 into the sleeve 121. The central portion 110₃ of the occlusion element 110 remains exposed.

The trailing anchor 120² further includes and a head 122 proximate a free axial end 120ₓ₁ of the trailing anchor 120², configured and arranged for releasable engagement by a pusher delivery catheter PdC for delivery of the mechanical tubal occlusion contraceptive device 100 into a fallopian tube FT.

The head 122 can include an approximately 1 mm diameter radial r through hole 129 configured and arranged to accommodate passage of a releasable tether T (e.g., a thin nitinol wire) extending from the distal tip PdCx₁ of a pusher delivery catheter PdC for securing the tip PdCx₁ of the catheter PdC to the head 122.

The mechanical tubal occlusion contraceptive device 100 preferably has an axial length of about 4 to 7 cm and a cross-sectional area of about 1 to 3 mm², with a 2 to 5 cm long exposed central portion 110₃. The anchors 120 are each preferably about 11 mm long and 1.5 mm in diameter.

The leading anchor 120¹ includes a plurality of radially r extending barbs 124 extending from a shaft 123 operable for resisting axial x movement of the leading anchor 120¹ relative to a fallopian tube FT into which the mechanical tubal occlusion contraceptive device 100 has been implanted. Similarly, the trailing anchor 120² includes a plurality of radially extending barbs 124 extending from a shaft 123 operable for resisting axial x movement of the trailing anchor 120² relative to a fallopian tube FT into which the mechanical tubal occlusion contraceptive device 100 has been implanted. The barbs 124 can be axially x spaced, radially r projecting, conical discs.

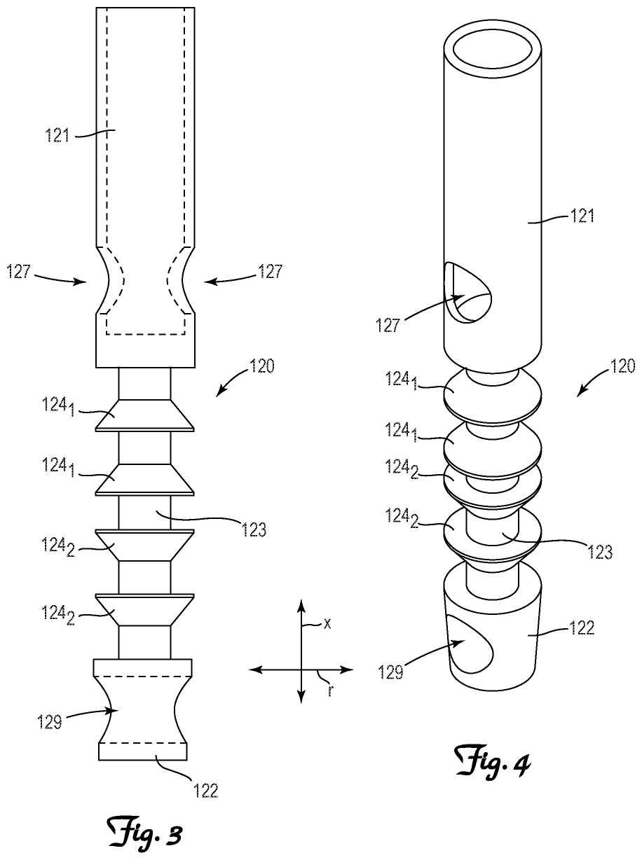

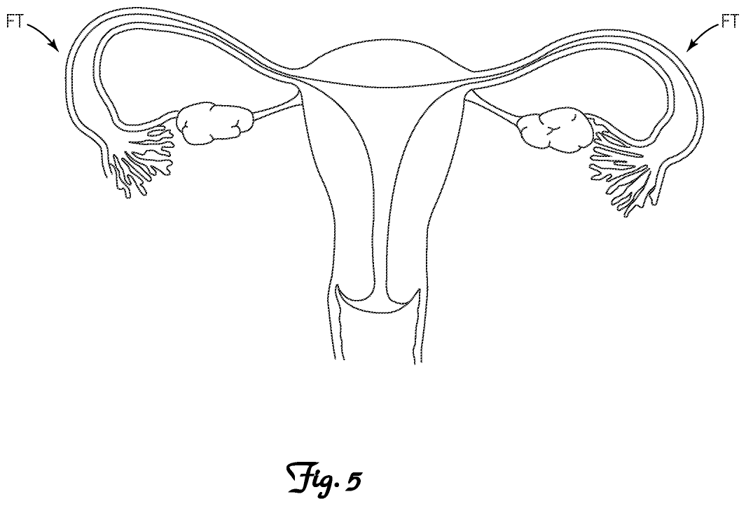

Preferably, at least one of the barbs 124 on each of the leading and trailing anchors 120¹ and 120² preferentially resists axial x movement of the mechanical tubal occlusion contraceptive device 100 in the first axial direction x₁ relative to a fallopian tube FT into which the mechanical tubal occlusion contraceptive device 100 has been implanted (i.e., barbs 124₁ depicted in FIGS. 3 and 4), and at least one of the barbs 124 on each of the leading and trailing anchors 120¹ and 120² preferentially resists axial x movement of the mechanical tubal occlusion contraceptive device 100 in the second axial direction x₂ relative to a fallopian tube FT into which the mechanical tubal occlusion contraceptive device 100 has been implanted (i.e., barbs 124₂ depicted in FIGS. 3 and 4).

As depicted in FIGS. 3 and 4, the anchors 120 can each include four sharp retention disks or barbs 124, so configured that two have the conical segments oriented towards one axial end 120ₓ₁ of the anchor 120 and two have the conical segments oriented towards the other axial end 120ₓ₂ of the anchor 120. Such orientation of the anchors 120 ensure that once the mechanical tubal occlusion contraceptive device 100 is placed in a fallopian tube FT elastic recoil of the fallopian tube FT around the anchor 120 will lock the anchor 120 in place. If all the retention disks or barbs 124 are oriented in the same direction, it is easy to pass it into the fallopian tube FT in one direction, but cannot be pulled out. However, having two each oriented as in FIGS. 3 and 4 locks the anchor 120 in place. It cannot be moved in either axial direction x₁ or x₂.

The barbs 124 on the leading anchor 120¹ preferably project radially r from a shaft 123 which projects in the first axial direction x₁ from the sleeve 121 of the leading anchor 120¹, and the barbs 124 on the trailing anchor 120² preferably project radially r from a shaft 123 which projects in the second axial direction x₂ from the sleeve 121 of the trailing anchor 120² to the head 122.

The leading and trailing anchors 120¹ and 120² can conveniently be mirror images of one another.

The first embodiment of the mechanical tubal occlusion contraceptive device 100 is capable of being deployed under radiographic control. The mechanical tubal occlusion contraceptive device 100 is a larger device passed through standard vascular sheaths S, rather than through the relatively small channels of a hysteroscope (not shown). For this reason the mechanical tubal occlusion contraceptive device 100 does not have to expand after delivery. Radial expansion of existing contraceptive occlusion devices post deployment leads to stretching of the fallopian tubes FT and has been the primary reason for pain and cramping in patients. Using angiographic catheters and interventional radiologic (IR) techniques permit superior results.

### Assembly

The first and second axial end portions 110₁ and 110₂ of the occlusion element 110 are each inserted into the sleeve 121 of a respective leading and trailing anchor 120¹ and 120² until the occlusion element 110 is visible in the inspection hole 127. The sleeve 121 of each anchor 120¹ and 120² is then crimped 126 with a mechanical crimper onto the occlusion element 110. This robustly secures an anchor 120 to each end of the occlusion element 110 to form the mechanical tubal occlusion contraceptive device 100.

The mechanical tubal occlusion contraceptive device 100 is assembled in a clean room, packaged and sterilized.

### Summary Implant Technique

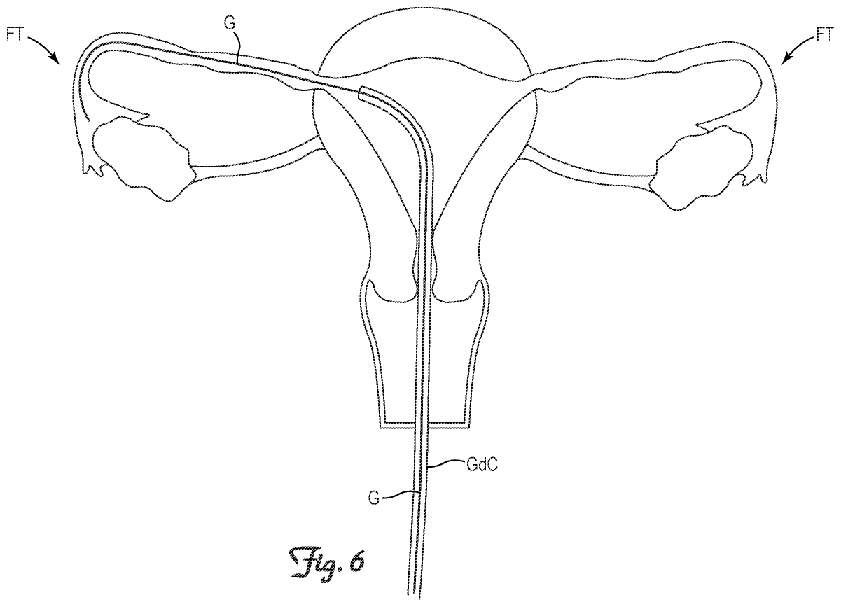

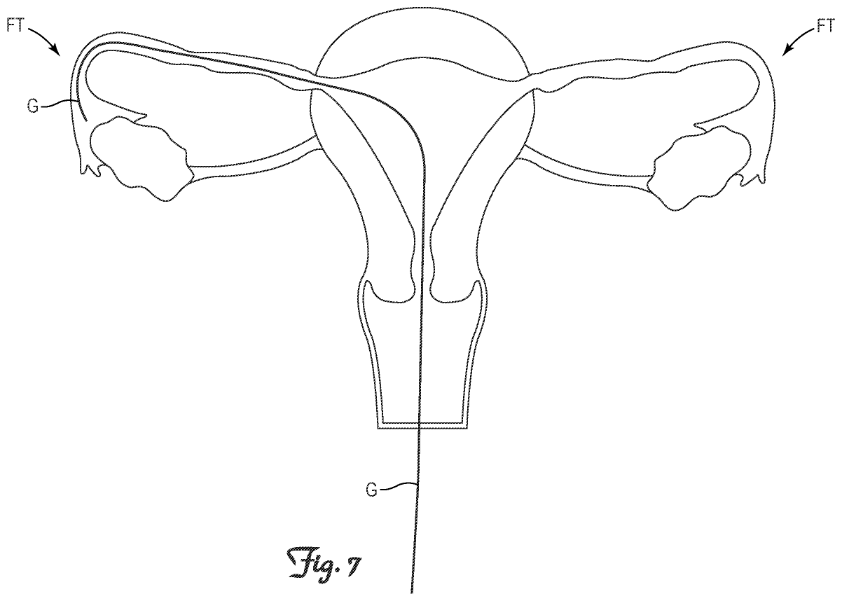

Referring to FIGS. 6 and 7, a guidewire G is transvaginally introduced via guidewire delivery catheter GdC into a fallopian tube FT and the guidewire delivery catheter GdC withdrawn.

Referring to FIGS. 8 and 9, a conical tipped vascular introducer sheath S is introduced into the fallopian tube FT over the guidewire G and both the distal tip and the guidewire G withdrawn.

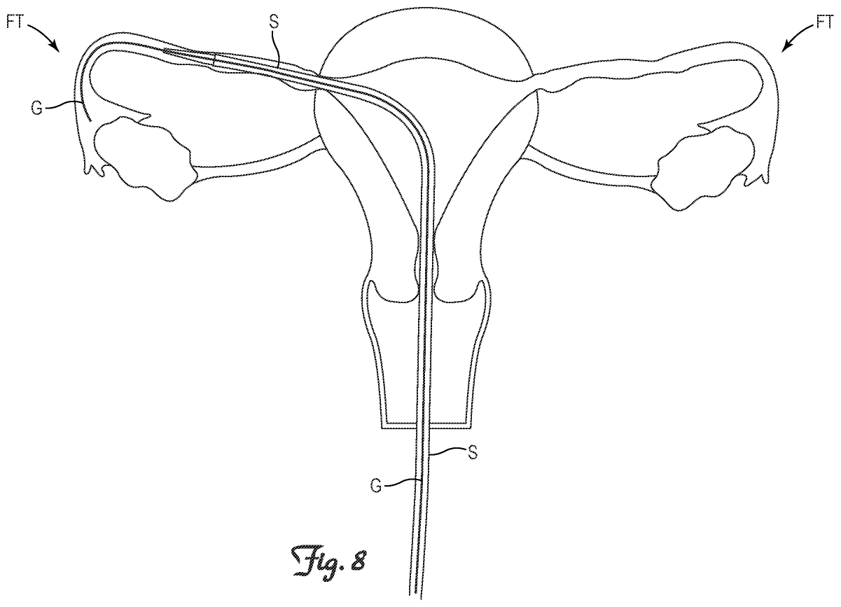

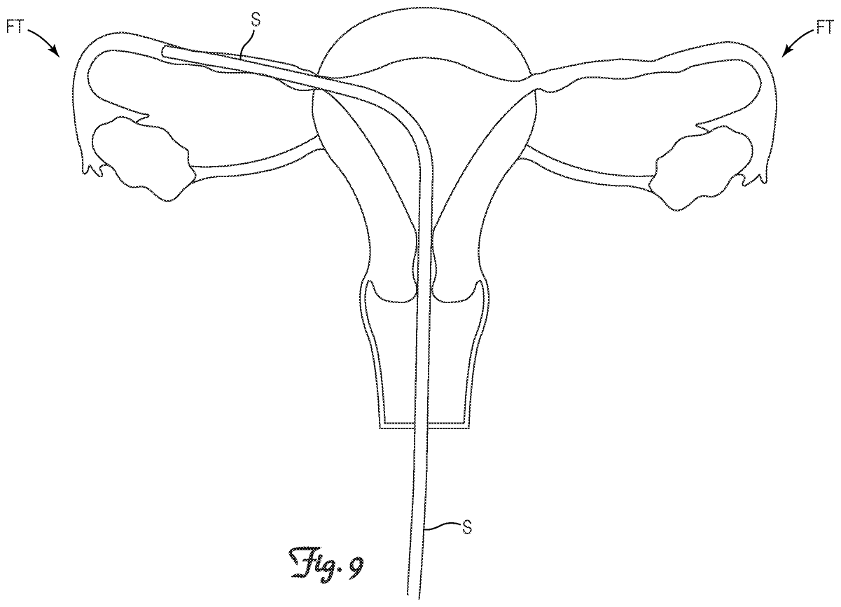

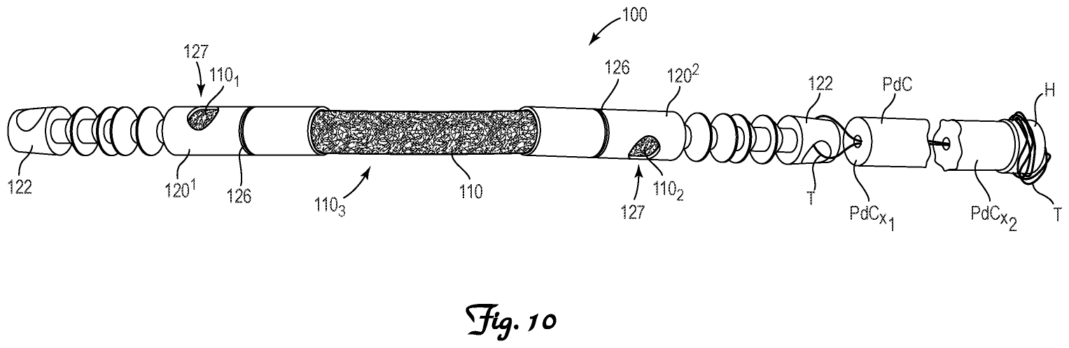

Referring to FIG. 10, the trailing anchor 120² is connected to a pusher delivery catheter PdC (e.g., 40 cm custom pusher delivery catheter). A tether T, such as a 110 cm long 0.005 inch diameter Nitinol wire, is passed through the radial hole 129 in the head 122 on the trailing anchor 120² until both ends of the tether T come together. The ends of the tether T are then passed through the lumen of the pusher delivery catheter PdC from the tip of the catheter PdCx₁ until they exit the hub H of the catheter PdC at the other end of the catheter PdCx₂. Both ends of the tether T are pulled taut such that the trailing anchor 120² is held snuggly against the tip of the catheter PdCx₁. The ends of the tether T are wrapped around the hub H and a threaded screw cap (not shown) is firmly screwed onto the hub H to lock the ends of the tether T into place.

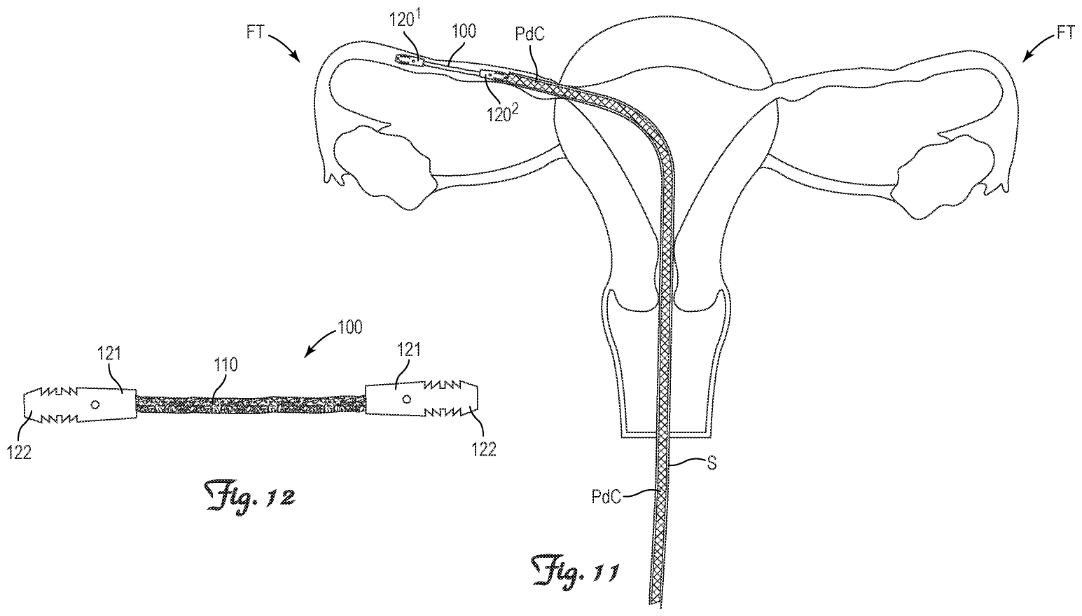

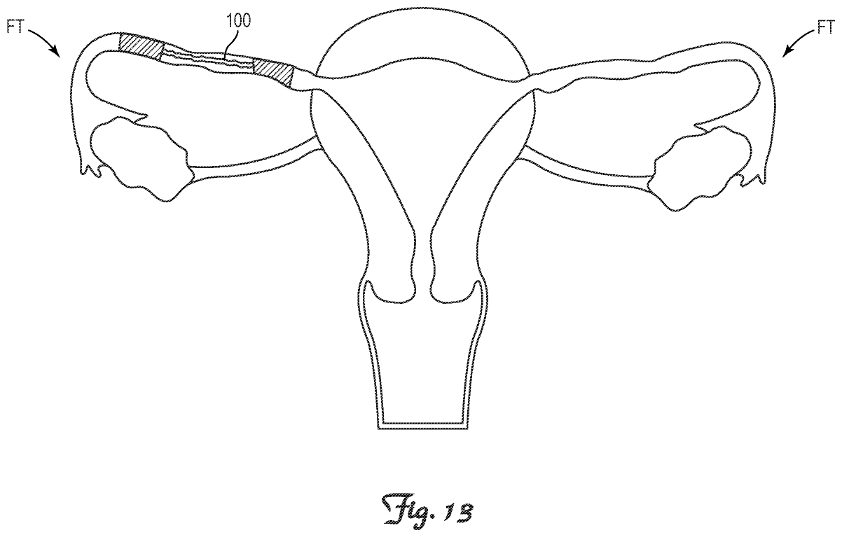

Referring to FIGS. 11-13, the assembled mechanical tubal occlusion contraceptive device 100, tethered to the pusher delivery catheter PdC, is axially x pushed through the lumen of the introducer sheath S by the pusher delivery catheter PdC until the entire assembled mechanical tubal occlusion contraceptive device 100 is positioned within the fallopian tube FT. The ends of the tether T wrapped around the hub H are then detached from one another and one end of the tether T pulled until the other end of the tether T is pulled through the radial hole 129 in the head 122 on the trailing anchor 120². The pusher delivery catheter PdC and the introducer sheath S are then withdrawn, leaving the mechanical tubal occlusion contraceptive device 100 within the fallopian tube FT.

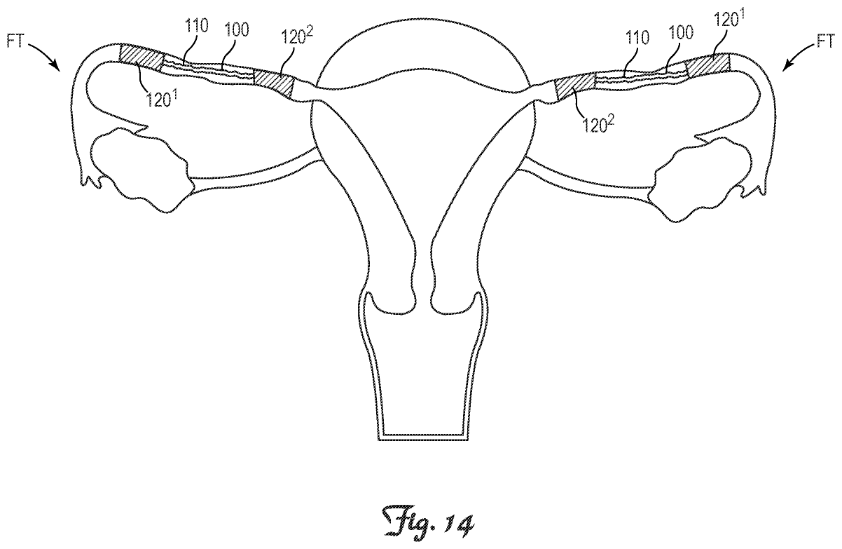

Referring to FIG. 14, the process is repeated for the other fallopian tube FT.

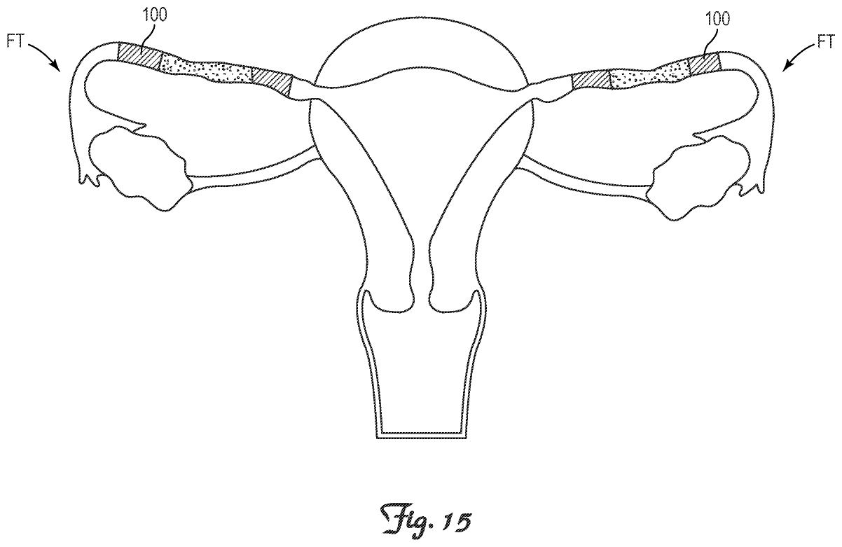

Referring to FIG. 15, scar tissue will form around the implanted mechanical tubal occlusion contraceptive devices 100, resulting in a permanent occlusion of both fallopian tubes FT.

### Second Embodiment

### Device

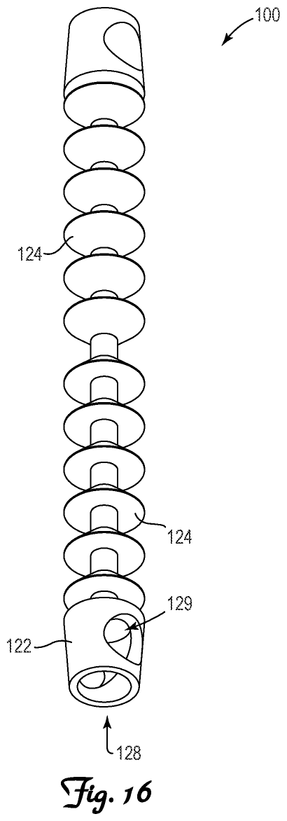

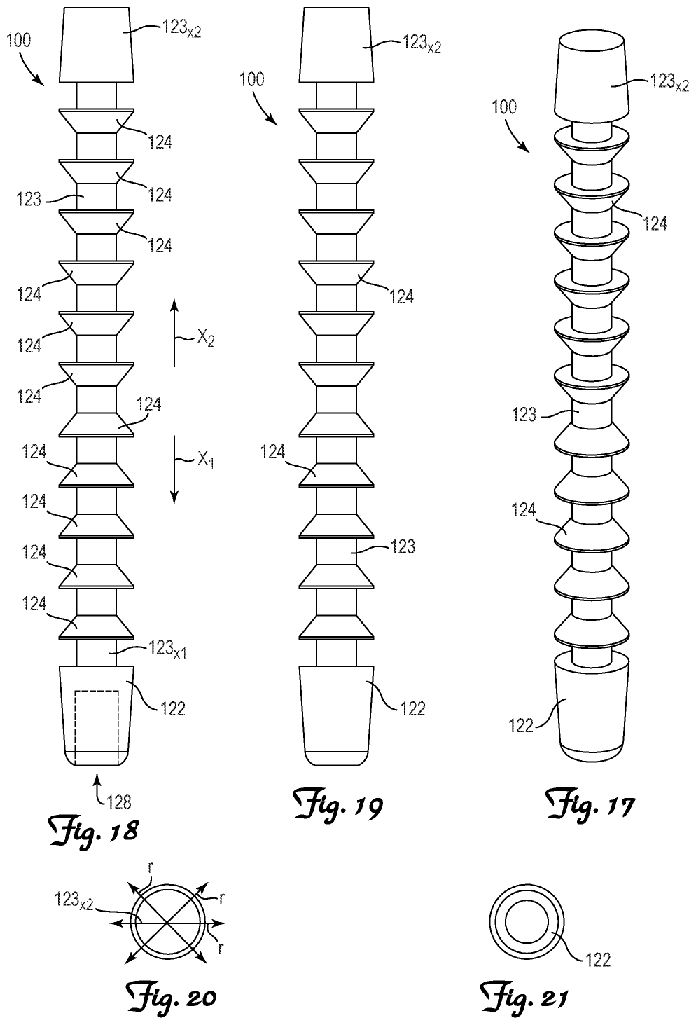

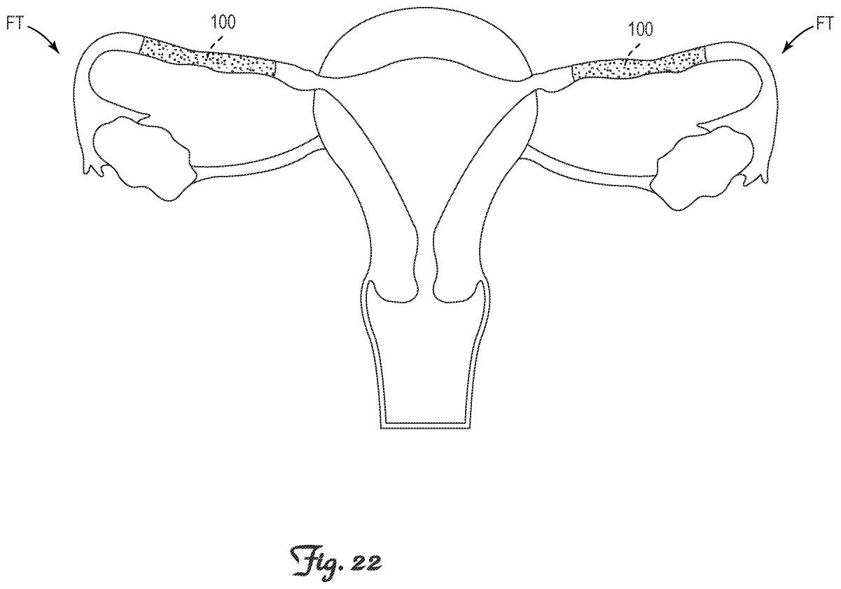

Referring to FIGS. 16-21, a second embodiment of a mechanical tubal occlusion contraceptive device 100 includes an axially elongated shaft 123, a head 122 and a plurality of barbs 124.

The shaft 123 has a proximal axial end 123ₓ₁ spaced in a first axial direction x₁ from a distal tip 123ₓ₂, and defines a second axial direction x₂ opposite the first axial direction x₁.

The head 122 projects in the first axial direction x₁ from the proximal axial end 123ₓ₁ of the shaft 123, and is configured and arranged for releasable engagement by a pusher delivery catheter PdC for delivery of the mechanical tubal occlusion contraceptive device 100 into a fallopian tube FT.

The plurality of barbs 124 extend radially r from the shaft 123 intermediate the head 122 and the distal tip 123ₓ₂ of the shaft 123, and collectively resist axial x movement of the mechanical tubal occlusion contraceptive device 100 in both the first and second axial directions x₁ and x₂ relative to a fallopian tube FT into which the mechanical tubal occlusion contraceptive device 100 has been implanted. The barbs 124 can be axially x spaced, radially r projecting, conical discs.

Preferably, at least one of the barbs 124 on each of the leading and trailing anchors 120¹ and 120² preferentially resists axial x movement of the mechanical tubal occlusion contraceptive device 100 in the first axial direction x₁ relative to a fallopian tube FT into which the mechanical tubal occlusion contraceptive device 100 has been implanted, and at least one of the barbs 124 on each of the leading and trailing anchors 120¹ and 120² preferentially resists axial x movement of the mechanical tubal occlusion contraceptive device 100 in the second axial direction x₂ relative to a fallopian tube FT into which the mechanical tubal occlusion contraceptive device 100 has been implanted.

As depicted in FIGS. 16-19, the second embodiment of a mechanical tubal occlusion contraceptive device 100 can include 11 or 12 sharp retention disks or barbs 124, so configured that half have the conical segments oriented towards one axial end 123ₓ₁ of the shaft 123 and the other half have the conical segments oriented towards the other axial end 123ₓ₂ of the shaft 123. Such orientation of the barbs 124 ensure that once the mechanical tubal occlusion contraceptive device 100 is placed in a fallopian tube FT elastic recoil of the fallopian tube FT around the mechanical tubal occlusion contraceptive device 100 will lock the mechanical tubal occlusion contraceptive device 100 in place, as it cannot be moved in either axial direction x₁ or x₂.

The entire second embodiment of the mechanical tubal occlusion contraceptive device 100 can be CNC micro machined from biocompatible metallic alloys. In the preferred embodiment the mechanical tubal occlusion contraceptive device 100 is machined from 316 L stainless steel alloy that has been used in human implants for decades in orthopedic screws, plates etc. and is biocompatible, or from solid rods of polymers such as PEEK (Polyether Ether Ketone). Alternatively, the second embodiment of the mechanical tubal occlusion contraceptive device 100 can be manufactured by injection molding a biocompatible polymer such as PET (Polyethylene Terephthalate). PET will promote scarring and make the mechanical tubal occlusion contraceptive device 100 a permanent implant. The polymer may have substances added to make it radiopaque as is commonly employed, including the addition of Tantalum powder, or may have Platinum marker bands incorporated.

The head 122 can include an approximately 1 mm diameter radial r through hole 129 configured and arranged to accommodate passage of a releasable tether T (e.g., a thin nitinol wire) extending from the distal tip PdCx₁ of a pusher delivery catheter PdC for securing the tip PdCx₁ of the catheter PdC to the head 122.

The second embodiment of the mechanical tubal occlusion contraceptive device 100 preferably has an axial length of about 1 to 4 cm and a radial cross-sectional area of about 0.8 to 7 mm².

The second embodiment of the mechanical tubal occlusion contraceptive device 100 is capable of being deployed under radiographic control. The mechanical tubal occlusion contraceptive device 100 is a larger device passed through standard vascular sheaths S, rather than through the relatively small channels of a hysteroscope (not shown). For this reason the mechanical tubal occlusion contraceptive device 100 does not have to expand after delivery. Radial expansion of existing contraceptive occlusion devices post deployment leads to stretching of the fallopian tubes FT and has been the primary reason for pain and cramping in patients. Using angiographic catheters and interventional radiologic (IR) techniques permit superior results.

### Summary Implant Technique

As with the first embodiment, the second embodiment of the mechanical tubal occlusion contraceptive device 100 is implanted by connecting a pusher delivery catheter PdC (e.g., 40 cm custom pusher delivery catheter) to the head 122 at one of the axial ends 123ₓ₁ or 123ₓ₂ of the shaft 123. A tether T, such as a 110 cm long 0.005 inch diameter Nitinol wire, is passed through the radial hole 129 in the head 122 until both ends of the tether T come together. The ends of the tether T are then passed through the lumen of the pusher delivery catheter PdC from the tip of the catheter PdCx₁ until they exit the hub H of the catheter PdC at the other end of the catheter PdCx₂. Both ends of the tether T are pulled taut such that the head 122 is held snuggly against the tip of the catheter PdCx₁. The ends of the tether T are wrapped around the hub H and a threaded screw cap (not shown) is firmly screwed onto the hub H to lock the ends of the tether T into place.

### Tubal Occlusion Contraceptive Appliance 200

The tubal occlusion contraceptive appliance 200 is used to occlude the cornual region of the uterine cavity and the proximal fallopian tube FT.

### First Embodiment

### Product

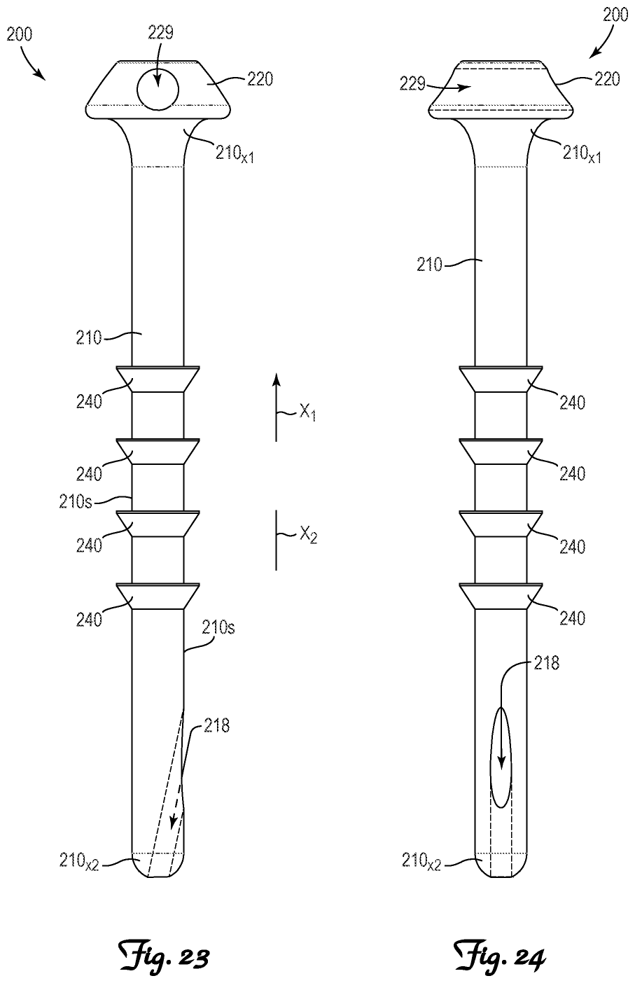

Referring to FIGS. 23 and 24, a first embodiment of a tubal occlusion contraceptive appliance 200 includes an axially elongated shaft 210, a cap 220, a plurality of barbs 240 and a guideway.

The shaft 210 has a proximal axial end 210ₓ₁ spaced in a first axial direction x₁ from a distal tip 210ₓ₂, and defines a second axial direction x₂ opposite the first axial direction x₁.

The shaft 210 preferably has an axial length of about 1 to 4 cm and a radial cross-sectional area of about 0.8 to 7 mm².

The cap 220 projects in the first axial direction x₁ from the proximal axial end 210ₓ₁ of the shaft 210, and is sized, configured and arranged to inhibit axial x travel of the cap 220 into a fallopian tube FT. The cap 220 should have a radial r diameter greater than the radial r diameter of the shaft 210 and a radial r diameter greater than the radial r diameter of each barb 240.

The plurality of barbs 240 extend radially r from the shaft 210 intermediate the cap 220 and the distal tip 210ₓ₂ of the shaft 210, and are configured and arranged to facilitate slide fit insertion of the shaft 210 into a fallopian tube FT in the second axial direction x₂ while preferentially resisting axial x movement of the shaft 210 in the first axial direction x₁ out from the fallopian tube FT once inserted. The barbs 240 can be axially x spaced, radially r projecting, conical discs.

The guideway is a distal guideway 218 that extends through the shaft 210 proximate the distal tip 210ₓ₂ of the shaft 210 for accommodating passage of a guidewire G for guiding insertion of the contraceptive appliance 200 into a fallopian tube FT.

The distal guideway 218 preferably angles inward in the second axial direction x₂ from a side 210s of the shaft 210 to the distal tip 210ₓ₂ of the shaft 210.

The entire first embodiment of the tubal occlusion contraceptive appliance 200 can be CNC micro machined from biocompatible metallic alloys. In the preferred embodiment the contraceptive appliance 200 is machined from 316 L stainless steel alloy that has been used in human implants for decades in orthopedic screws, plates etc. and is biocompatible, or from solid rods of polymers such as PEEK (Polyether Ether Ketone). Alternatively, the first embodiment of the tubal occlusion contraceptive appliance 200 can be manufactured by injection molding a biocompatible polymer such as PET (Polyethylene Terephthalate). PET will promote scarring and make the contraceptive appliance 200 a permanent implant. The polymer may have substances added to make it radiopaque as is commonly employed, including the addition of Tantalum powder, or may have Platinum marker bands incorporated.

The cap 220 can include an approximately 1 mm diameter radial r through hole 229 configured and arranged to accommodate passage of a releasable tether T (e.g., a thin nitinol wire) extending from the distal tip PdCx₁ of a pusher delivery catheter PdC for securing the tip PdCx₁ of the catheter PdC to the cap 220.

### Summary Implant Technique

Referring to FIG. 25, a guidewire G is transvaginally introduced via a guidewire delivery catheter GdC into a fallopian tube FT and the guidewire delivery catheter GdC withdrawn.

The cap 220 is connected to a pusher delivery catheter PdC (e.g., 40 cm custom pusher delivery catheter). A tether T, such as a 110 cm long 0.005 inch diameter Nitinol wire, is passed through the radial hole 229 in the cap 220 until both ends of the tether T come together. The ends of the tether T are then passed through the lumen of the pusher delivery catheter PdC from the tip of the catheter PdCx₁ until they exit the hub H of the catheter PdC at the other end of the catheter PdCx₂. Both ends of the tether T are pulled taut such that the trailing anchor 120² is held snuggly against the tip of the catheter PdCx₁. The ends of the tether T are wrapped around the hub H and a threaded screw cap (not shown) is firmly screwed onto the hub H to lock the ends of the tether T into place.

Referring to FIG. 26, the free end of the guidewire G is fed through the distal guideway 218 in the shaft 210 and the tethered tubal occlusion contraceptive appliance 200 pushed along the guidewire G by the pusher delivery catheter PdC until the shaft 210 is positioned within the fallopian tube FT and the cap 220 is seated against the opening from the fallopian tube FT to the uterus. The ends of the tether T wrapped around the hub H are then detached from one another and one end of the tether T pulled until the other end of the tether T is pulled through the radial hole 229 in the cap 220. The pusher delivery catheter PdC and the guidewire G are then withdrawn, leaving the shaft 210 of the tubal occlusion contraceptive appliance 200 within the fallopian tube FT.

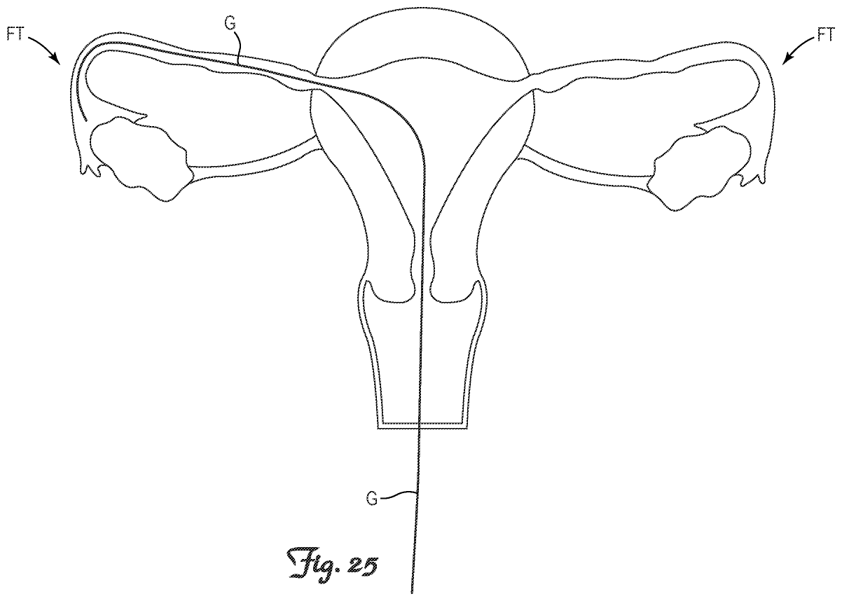

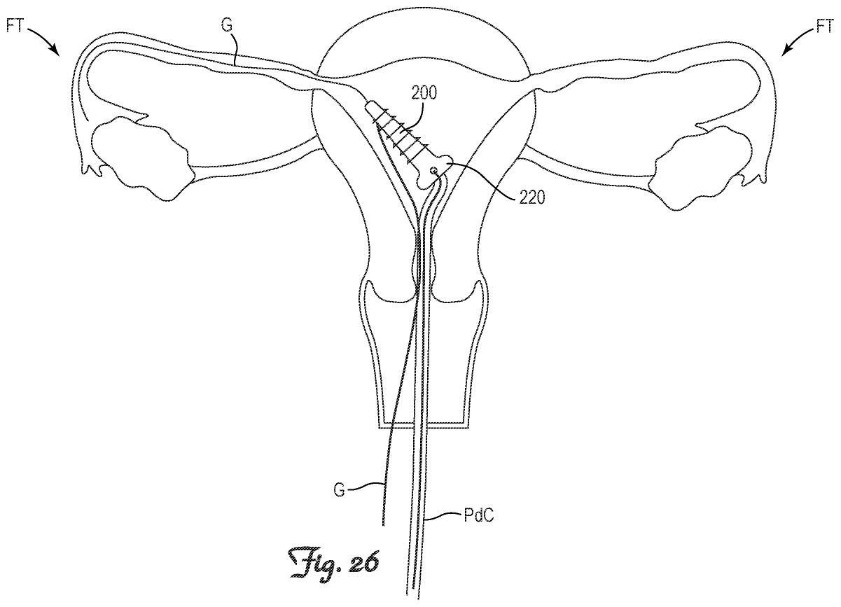

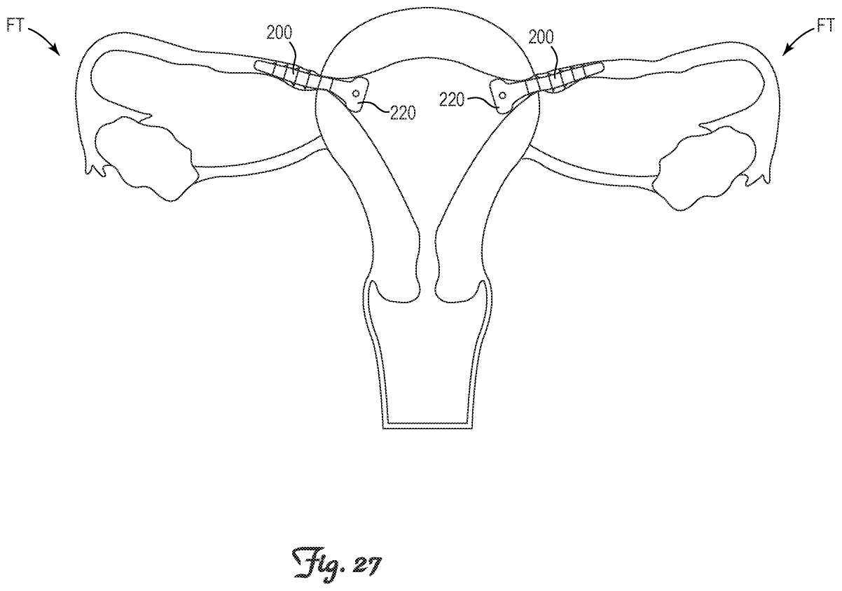

Referring to FIG. 27, the process is repeated for the other fallopian tube FT.

### Second Embodiment

### Product

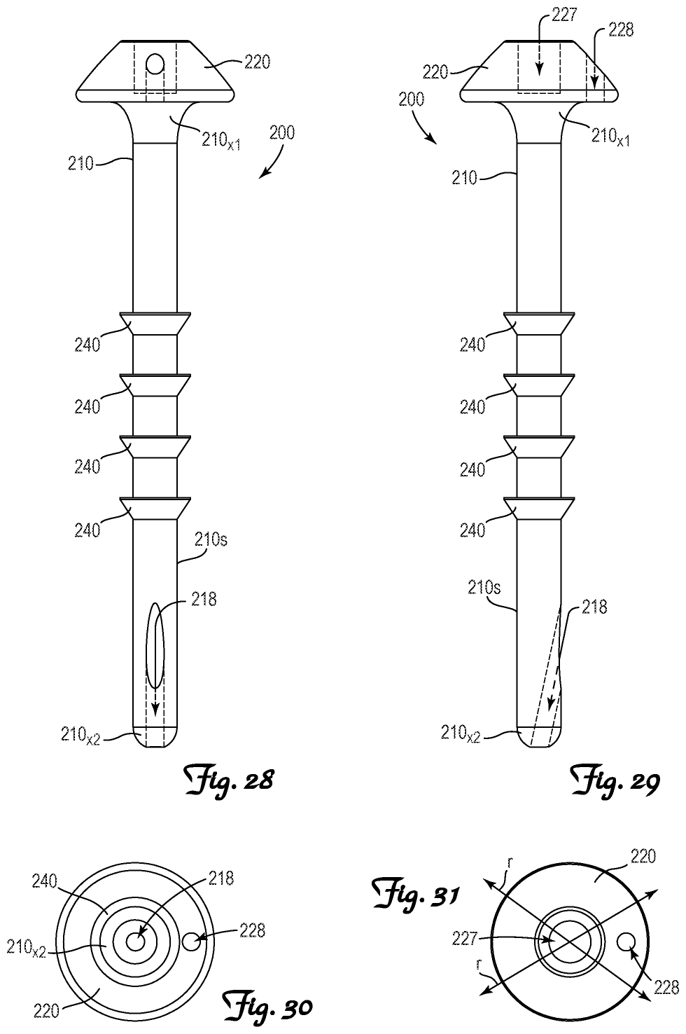

Referring to FIGS. 28-31, a second embodiment of a tubal occlusion contraceptive appliance 200 includes an axially elongated shaft 210, a cap 220, a plurality of barbs 240 and a dual passage guideway.

The shaft 210 has a proximal axial end 210ₓ₁ spaced in a first axial direction x₁ from a distal tip 210ₓ₂, and defines a second axial direction x₂ opposite the first axial direction x₁.

The shaft 210 preferably has an axial length of about 1 to 4 cm and a radial cross-sectional area of about 0.8 to 7 mm².

The cap 220 projects in the first axial direction x₁ from the proximal axial end 210ₓ₁ of the shaft 210, and is sized, configured and arranged to inhibit axial x travel of the cap 220 into a fallopian tube FT. The cap 220 should have a radial r diameter greater than the radial r diameter of the shaft 210 and a radial r diameter greater than the radial r diameter of each barb 240.

The plurality of barbs 240 extend radially r from the shaft 210 intermediate the cap 220 and the distal tip 210ₓ₂ of the shaft 210, and are configured and arranged to facilitate slide fit insertion of the shaft 210 into a fallopian tube FT in the second axial direction x₂ while preferentially resisting axial x movement of the shaft 210 in the first axial direction x₁ out from the fallopian tube FT once inserted. The barbs 240 can be axially x spaced, radially r projecting, conical discs.

The dual passage guideway includes an axially x extending proximal guideway 228 through the cap 220 and an axially x extending distal guideway 218 that extends through the shaft 210 proximate the distal tip 210ₓ₂ of the shaft 210. Both are configured and arranged to accommodate passage of a guidewire G for guiding insertion of the contraceptive appliance 200 into a fallopian tube FT.

The proximal guideway 228 is preferably in radially r spaced relationship from the shaft 210, while the distal guideway 218 preferably angles inward in the second axial direction x₂ from a side 210s of the shaft 210 to the distal tip 210ₓ₂ of the shaft 210.

The entire first embodiment of the tubal occlusion contraceptive appliance 200 can be CNC micro machined from biocompatible metallic alloys. In the preferred embodiment the contraceptive appliance 200 is machined from 316 L stainless steel alloy that has been used in human implants for decades in orthopedic screws, plates etc. and is biocompatible, or from solid rods of polymers such as PEEK (Polyether Ether Ketone). Alternatively, the first embodiment of the tubal occlusion contraceptive appliance 200 can be manufactured by injection molding a biocompatible polymer such as PET (Polyethylene Terephthalate). PET will promote scarring and make the contraceptive appliance 200 a permanent implant. The polymer may have substances added to make it radiopaque as is commonly employed, including the addition of Tantalum powder, or may have Platinum marker bands incorporated.

The cap 220 can include an internally threaded axial orifice 227 open opposite the shaft 210 for threadable engagement with the threaded distal tip PdCx₁ of a pusher delivery catheter PdC for securing the tip PdCx₁ of the catheter PdC to the cap 220.

### Summary Implant Technique

A guidewire G is transvaginally introduced via a guidewire delivery catheter GdC into a fallopian tube FT and the guidewire delivery catheter GdC withdrawn.

The cap 220 is threadably attached to a pusher delivery catheter PdC (e.g., 40 cm custom pusher delivery catheter).

The free end of the guidewire G is fed through the distal guideway 218 in the shaft 210 and then through the proximal guideway 228 in the cap 220. The attached tubal occlusion contraceptive appliance 200 is pushed along the guidewire G by the pusher delivery catheter PdC until the shaft 210 is positioned within the fallopian tube FT and the cap 220 is seated against the opening from the fallopian tube FT to the uterus. The pusher delivery catheter PdC is rotated about its axial axis to threadably disengage from tubal occlusion contraceptive appliance 200, and the disengaged pusher delivery catheter PdC and the guidewire G withdrawn, leaving the shaft 210 of the tubal occlusion contraceptive appliance 200 within the fallopian tube FT.

The process is repeated for the other fallopian tube FT.

### Third Embodiment

### Product

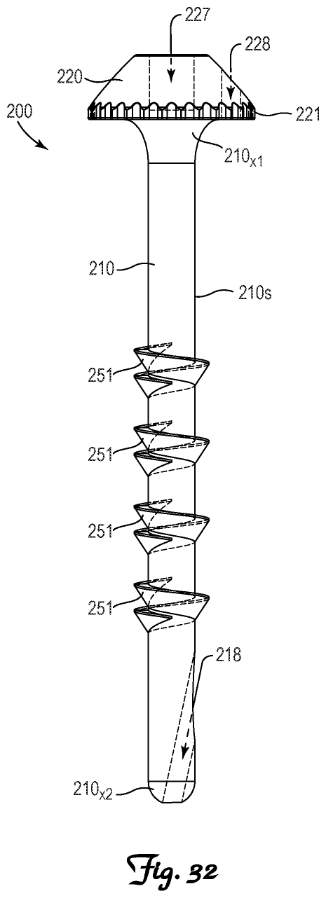

Referring to FIG. 32, a third embodiment of a tubal occlusion contraceptive appliance 200 includes an axially elongated shaft 210, a cap 220, a plurality of screw thread segment 251 and a dual passage guideway.

The shaft 210 has a proximal axial end 210ₓ₁ spaced in a first axial direction xi from a distal tip 210ₓ₂, and defines a second axial direction x₂ opposite the first axial direction x₁.

The shaft 210 preferably has an axial length of about 1 to 4 cm and a radial cross-sectional area of about 0.8 to 7 mm².

The cap 220 projects in the first axial direction x₁ from the proximal axial end 210ₓ₁ of the shaft 210, and is sized, configured and arranged to inhibit axial x travel of the cap 220 into a fallopian tube FT. The cap 220 should have a radial r diameter greater than the radial r diameter of the shaft 210 and a radial r diameter greater than the radial r diameter of each screw thread segment 251.

The plurality of screw thread segment 251 extend radially r from the shaft 210 intermediate the cap 220 and the distal tip 210ₓ₂ of the shaft 210, and are configured and arranged to (i) facilitate slide fit insertion of the shaft 210 into a fallopian tube FT in the second axial direction x₂ with or without rotation of the contraceptive appliance 200 in a first direction while preferentially resisting sliding axial x movement of the shaft 210 in the first axial direction x₁ out from the fallopian tube FT once inserted, and (ii) effect withdrawal of the tubal occlusion contraceptive appliance 200 from the fallopian tube FT upon rotation of the contraceptive appliance 200 in a second direction opposite the first direction. The plurality of screw thread segments 251 are discrete, axially spaced, screw threads, each having a limited number of turns, preferably between about 1 to 2 turns.

The dual passage guideway includes an axially x extending proximal guideway 228 through the cap 220 and an axially x extending distal guideway 218 that extends through the shaft 210 proximate the distal tip 210ₓ₂ of the shaft 210. Both are configured and arranged to accommodate passage of a guidewire G for guiding insertion of the contraceptive appliance 200 into a fallopian tube FT.

The proximal guideway 228 is preferably in radially r spaced relationship from the shaft 210, while the distal guideway 218 preferably angles inward in the second axial direction x₂ from a side 210s of the shaft 210 to the distal tip 210ₓ₂ of the shaft 210.

The entire first embodiment of the tubal occlusion contraceptive appliance 200 can be CNC micro machined from biocompatible metallic alloys. In the preferred embodiment the contraceptive appliance 200 is machined from 316 L stainless steel alloy that has been used in human implants for decades in orthopedic screws, plates etc. and is biocompatible, or from solid rods of polymers such as PEEK (Polyether Ether Ketone). Alternatively, the first embodiment of the tubal occlusion contraceptive appliance 200 can be manufactured by injection molding a biocompatible polymer such as PET (Polyethylene Terephthalate). PET will promote scarring and make the contraceptive appliance 200 a permanent implant. The polymer may have substances added to make it radiopaque as is commonly employed, including the addition of Tantalum powder, or may have Platinum marker bands incorporated.

The cap 220 can include an internally threaded axial orifice 227 open opposite the shaft 210 for threadable engagement with the threaded distal tip PdCx₁ of a pusher delivery catheter PdC for securing the tip PdCx₁ of the catheter PdC to the cap 220.

An exterior circumferential surface 221 of the cap 220 is preferably knurled to facilitate gripping and rotation of the cap 220 with a forceps (not shown) to effect withdrawal of the tubal occlusion contraceptive appliance 200 from a fallopian tube FT after insertion.

### Summary Implant Technique

A guidewire G is transvaginally introduced via a guidewire delivery catheter GdC into a fallopian tube FT and the guidewire delivery catheter GdC withdrawn.

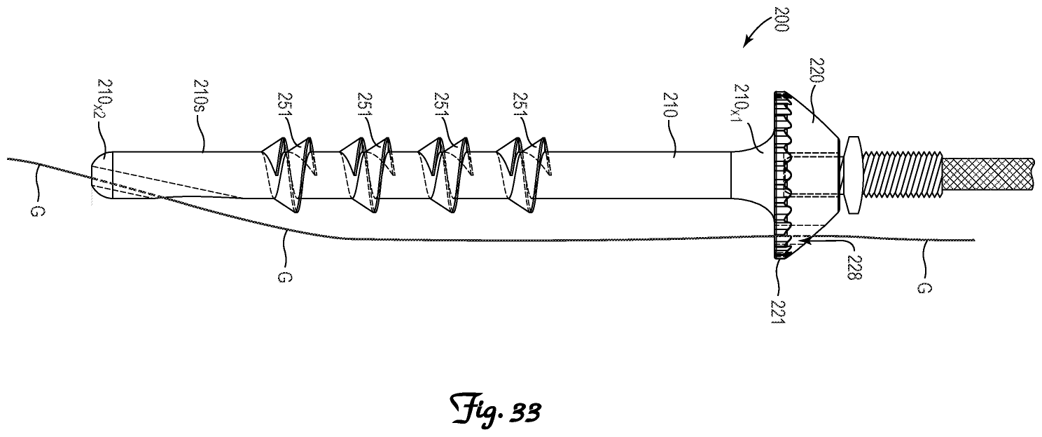

Referring to FIG. 33, the cap 220 is threadably attached to a pusher delivery catheter PdC (e.g., 40 cm custom pusher delivery catheter).

The free end of the guidewire G is fed through the distal guideway 218 in the shaft 210 and then through the proximal guideway 228 in the cap 220. The attached tubal occlusion contraceptive appliance 200 is pushed along the guidewire G by the pusher delivery catheter PdC, with or without insertional rotation of the contraceptive appliance 200 about its axial x axis, until the shaft 210 is positioned within the fallopian tube FT and the cap 220 is seated against the opening from the fallopian tube FT to the uterus. The pusher delivery catheter PdC is rotated about its axial x axis to threadably disengage from tubal occlusion contraceptive appliance 200, and the disengaged pusher delivery catheter PdC and the guidewire G withdrawn, leaving the shaft 210 of the tubal occlusion contraceptive appliance 200 within the fallopian tube FT. If desired, at any time prior to disengagement of the pusher delivery catheter PdC from the tubal occlusion contraceptive appliance 200, the pusher delivery catheter PdC can be rotated about its axial x axis in the threadable engagement direction to effect rotation of the entire tubal occlusion contraceptive appliance 200 in a direction that effects threaded withdrawal of the tubal occlusion contraceptive appliance 200 out from the fallopian tube FT.

The process is repeated for the other fallopian tube FT.

### Fourth Embodiment

### Product

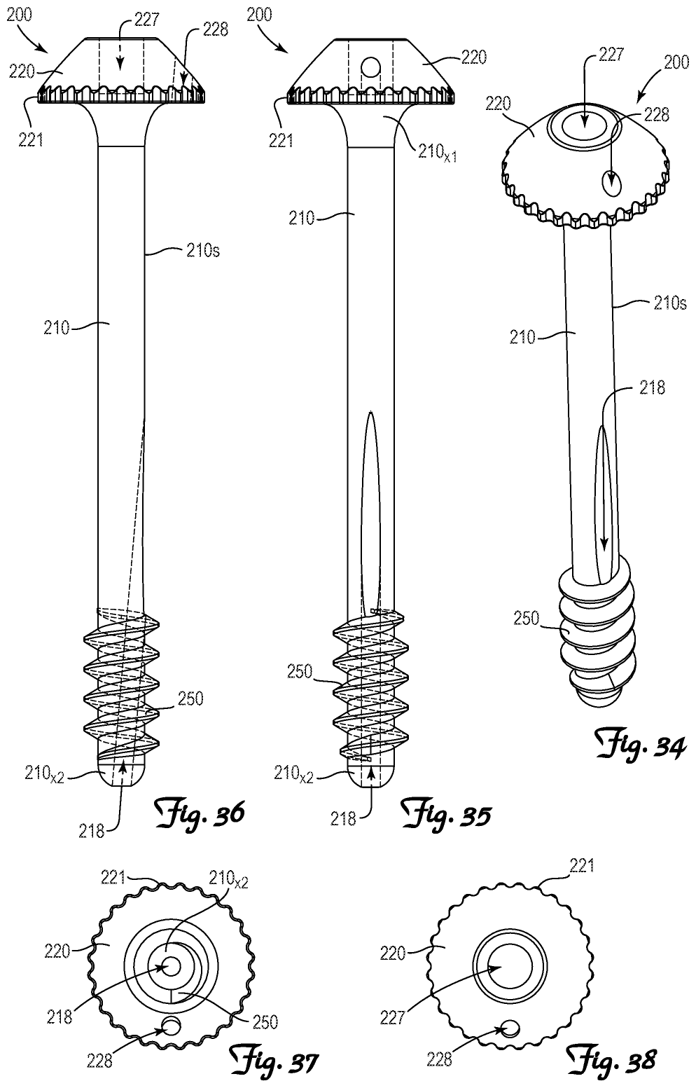

Referring to FIGS. 34-38, a fourth embodiment of a tubal occlusion contraceptive appliance 200 includes an axially elongated shaft 210, a cap 220, screw threads 250 and a dual passage guideway.

The shaft 210 has a proximal axial end 210ₓ₁ spaced in a first axial direction xi from a distal tip 210ₓ₂, and defines a second axial direction x₂ opposite the first axial direction x₁.

The shaft 210 preferably has an axial length of about 1 to 4 cm and a radial cross-sectional area of about 0.8 to 7 mm².

The cap 220 projects in the first axial direction x₁ from the proximal axial end 210ₓ₁ of the shaft 210, and is sized, configured and arranged to inhibit axial x travel of the cap 220 into a fallopian tube FT. The cap 220 should have a radial r diameter greater than the radial r diameter of the shaft 210 and a radial r diameter greater than the radial r diameter of each screw thread segment 251.

The screw threads 250 extend radially r around the shaft 210 for effecting axial x insertion of the tubal occlusion contraceptive appliance 200 into a fallopian tube FT upon rotation of the contraceptive appliance 200 about its axial x axis in a first rotational direction, and withdrawal of the tubal occlusion contraceptive appliance 200 from the fallopian tube FT upon rotation of the contraceptive appliance 200 about its axial x axis in a second rotational direction opposite the first direction. The screw threads 250 include several turns.

The dual passage guideway includes an axially x extending proximal guideway 228 through the cap 220 and an axially x extending distal guideway 218 that extends through the shaft 210 proximate the distal tip 210ₓ₂ of the shaft 210. Both are configured and arranged to accommodate passage of a guidewire G for guiding insertion of the contraceptive appliance 200 into a fallopian tube FT.

The proximal guideway 228 is preferably in radially r spaced relationship from the shaft 210, while the distal guideway 218 preferably angles inward in the second axial direction x₂ from a side 210s of the shaft 210 to the distal tip 210ₓ₂ of the shaft 210.

The central axis of the proximal guideway 228 and distal guideway 218 preferably extend along a common line.

The entire first embodiment of the tubal occlusion contraceptive appliance 200 can be CNC micro machined from biocompatible metallic alloys. In the preferred embodiment the contraceptive appliance 200 is machined from 316 L stainless steel alloy that has been used in human implants for decades in orthopedic screws, plates etc. and is biocompatible, or from solid rods of polymers such as PEEK (Polyether Ether Ketone). Alternatively, the first embodiment of the tubal occlusion contraceptive appliance 200 can be manufactured by injection molding a biocompatible polymer such as PET (Polyethylene Terephthalate). PET will promote scarring and make the contraceptive appliance 200 a permanent implant. The polymer may have substances added to make it radiopaque as is commonly employed, including the addition of Tantalum powder, or may have Platinum marker bands incorporated.

The cap 220 can include an internally threaded axial orifice 227 open opposite the shaft 210 for threadable engagement with the threaded distal tip PdCx₁ of a pusher delivery catheter PdC for securing the tip PdCx₁ of the catheter PdC to the cap 220.

An exterior circumferential surface 221 of the cap 220 is preferably knurled to facilitate gripping and rotation of the cap 220 with a forceps (not shown) to effect withdrawal of the tubal occlusion contraceptive appliance 200 from a fallopian tube FT after insertion.

### Summary Implant Technique

A guidewire G is transvaginally introduced via a guidewire delivery catheter GdC into a fallopian tube FT and the guidewire delivery catheter GdC withdrawn.

The cap 220 is threadably attached to a pusher delivery catheter PdC (e.g., 40 cm custom pusher delivery catheter).

The free end of the guidewire G is fed through the distal guideway 218 in the shaft 210 and then through the proximal guideway 228 in the cap 220. The attached tubal occlusion contraceptive appliance 200 is pushed along the guidewire G by the pusher delivery catheter PdC until the screw threads 250 engage the sidewalls of the fallopian tube FT. Thereafter, the pusher delivery catheter PdC is rotated so as to effect insertional rotation of the contraceptive appliance 200 about its axial x axis. The contraceptive appliance 200 is rotated until the shaft 210 is positioned within the fallopian tube FT and the cap 220 is seated against the opening from the fallopian tube FT to the uterus. The pusher delivery catheter PdC is rotated about its axial x axis to threadably disengage from tubal occlusion contraceptive appliance 200, and the disengaged pusher delivery catheter PdC and the guidewire G withdrawn, leaving the shaft 210 of the tubal occlusion contraceptive appliance 200 within the fallopian tube FT. If desired, at any time prior to disengagement of the pusher delivery catheter PdC from the tubal occlusion contraceptive appliance 200, the pusher delivery catheter PdC can be rotated about its axial x axis in the threadable engagement direction to effect rotation of the entire tubal occlusion contraceptive appliance 200 in a direction that effects threaded withdrawal of the tubal occlusion contraceptive appliance 200 out from the fallopian tube FT.

The process is repeated for the other fallopian tube FT.

An implanted tubal occlusion contraceptive appliance 200 can be removed after implant by gripping the knurled circumferential surface 221 of the cap 220 with a forceps, rotating the cap 220 with the forceps about the axial x axis of the implanted tubal occlusion contraceptive appliance 200 whereby the screw threads 250 or screw thread segments 251 on the implanted tubal occlusion contraceptive appliance 200 effect axial x travel of the implanted tubal occlusion contraceptive appliance 200 completely out from the fallopian tube FT, and then removing the tubal occlusion contraceptive appliance 200 completely from the body.

### (Exemplary Preferred Device)

In its preferred embodiment, the tubal occlusion contraceptive appliance 200 is about 19 mm in length with a main shaft 210 about 1.25 mm in diameter. There are 4 retention disks 240 that are 2 mm in diameter each, located at the mid region of the shaft 210. The cap 220 is about 3.5 mm in diameter. There is a 1 mm hole 229 machined radially r across the cap 220, to permit it to be attached to the tether T of a delivery system such as a pusher delivery catheter PdC. At the distal tip 210ₓ₂ of the shaft 210 there is a machined angled distal guideway 218, that permits a 0.018″ guidewire G to be passed through it. The retention disks 240 are all oriented with their conical faces towards the distal tip 210ₓ₂ of the shaft 210. This permits the tubal occlusion contraceptive appliance 200 to be passed in the second axial direction x₂ in a fallopian tube FT, and then due to the elasticity of the fallopian tube FT and the orientation of the retention disks 240 is locked in place and will not pull out of the fallopian tube FT. The oversize nature of the cap 220, prevents the tubal occlusion contraceptive appliance 200 from migrating into the fallopian tube FT.

### Deployment Protocol

### Exemplary Pre-Deployment Protocol

The procedure can be performed in a radiology suite that permits deployment under state of the art fluoroscopy and cine-radiography. The patient should be screened with a blood test to rule out early pregnancy. A long term subcutaneous hormonal implant would preferably have been implanted in the weeks prior to the procedure or the patient will be on birth control pills to prevent a pregnancy in the first 3 months after the implant (till such time a repeat hysterosalpingography (HSG) confirms bilateral tubal occlusion). The patient should be provided with materials explaining the risks and benefits of the procedure and an informed consent obtained.

The procedure can be performed by a physician team consisting of an Interventional radiologist and a gynecologist.

The patient will be prepped and draped for the procedure. An IV access should be obtained and normal saline infused at a slow rate to ensure access. The patient should be sedated with 50 microgram bolus of Fentanyl and 0.5 mg Atropine injected to prevent bradycardia and a vasovagal reaction. The vagina and cervix should be cleansed with povidone-iodine using soaked sterile sponges on a long ring forceps. 1 to 2 ml of lidocaine should be intracervically injected, noting the presence of a wheal on the cervix, usually at the 6 and 12 o'clock positions where the tenaculum will be placed. A four quadrant cervical block can be undertaken by injecting 20 cc of 1% lidocaine without epinephrine at 4 spots around the cervix at 2, 4, 8 and 10 o'clock positions. The cervix can be held with a tenaculum, and a 6 French hystero-salpingography (HSG) catheter passed through the Os of the cervix into the uterine cavity. The balloon inflated, traction on the catheter maintained, and a standard HSG performed with a few ml of radiographic contrast injected into the uterine cavity till the fallopian tubes are also filled. The radiographs can be reviewed to confirm that both tubes are patent.

The HSG catheter can then be removed.

### Exemplary Deployment Protocol

### Mechanical Tubal Occlusion Contraceptive Device 100

Referring to FIGS. 6-9, 11 and 13-15, a 6 Fr. Short JR 4 (Judkins Right Coronary 4) guidewire delivery catheter GdC is passed into the uterus. Under fluoroscopy control and using the HSG image as a road map, the right fallopian tube FT is cannulated. One ml or two of non-ionic radiographic contrast is injected to selectively visualize the fallopian tube FT. An 80 cm soft tip, 0.018″ guidewire G is passed into the fallopian tube FT and the guidewire G advanced until it exits the fimbriated end of the fallopian tube FT. The guidewire delivery catheter GdC is withdrawn under fluoroscopic guidance, leaving the guidewire G in place.

A custom 35 cm, 5 Fr flexible introducer sheath S with dilator is passed over the guidewire G until the tip of the sheath S is at least 6 to 7 cms beyond the ostium of the fallopian tube FT. Once confirmed, the dilator and guidewire G are withdrawn, leaving the sheath S in place. The side arm is aspirated and the sheath S gently flushed with sterile normal saline.

The mechanical tubal occlusion contraceptive device 100 with its pusher delivery catheter PdC is removed from its sterile packaging. The hemostatic valve is disconnected from the 5 Fr. sheath S. The tip of the mechanical tubal occlusion contraceptive device 100 is placed into the sheath S and gently advanced until the pusher delivery catheter PdC enters the 5 Fr. sheath S. The pusher delivery catheter PdC is advanced into the sheath S under fluoroscopy. The rigid portions of the mechanical tubal occlusion contraceptive device 100, is easily visible under fluoroscopy as they are made from 316 L SS, or from solid rods of polymers such as PEEK (Polyether Ether Ketone). The pusher delivery catheter PdC is advanced until the leading anchor 120¹ reaches the tip of the sheath S.

Visually confirm that the entire mechanical tubal occlusion contraceptive device 100 is beyond the ostium of the fallopian tube FT and junction with the uterine cavity. The mechanical tubal occlusion contraceptive device 100 is, at this point, ready for deployment. Holding the pusher delivery catheter PdC firmly with one hand, the sheath S is slowly backed out with the other hand until the tip of the sheath S comes just proximal to the trailing anchor 120². It is desirable at this time to watch carefully for 1 to 2 minutes for the fallopian tube FT tone to return and for it to clamp down around the mechanical tubal occlusion contraceptive device 100. Gentle traction backwards over 1 to 2 mm will confirm this. This then is the stage to release the mechanical tubal occlusion contraceptive device 100. The screw cap over the hub H of the pusher delivery catheter PdC is unscrewed and placed to a side. The double 0.005″ Nitinol wire tether T is unwound from the hub H and the two are separated. Holding one wire firmly the other is gently pulled back till the first end goes into the lumen. The wire is continued to be gently pulled out. At all times the pusher delivery catheter PdC is held firmly with one hand and the hand should rest on the procedure table so that there is no traction on the pusher delivery catheter PdC. Once the Nitinol wire is completely removed, the mechanical tubal occlusion contraceptive device 100 is released.

The 5 Fr. custom introducer sheath S and the pusher delivery catheter PdC are removed. This completes deployment for one fallopian tube FT.

The same procedure is repeated for the other fallopian tube FT. The 6 Fr. JR 4 guidewire delivery catheter GdC is passed into the uterus and the other fallopian tube FT is cannulated. The other fallopian tube FT is wired G. The 5 Fr. introducer sheath S is passed into the other fallopian tube FT. The dilator and guidewire G are removed. A second mechanical tubal occlusion contraceptive device 100 is passed into the introducer sheath S and deployed in similar fashion.

After the second mechanical tubal occlusion contraceptive device 100 is deployed, no HSG should be performed because of concerns that the high pressure injection of contrast may dislodge or push the mechanical tubal occlusion contraceptive device 100 outwards in the fallopian tube FT.

The patient should be observed for a couple of hours and a plain Xray of the pelvis performed to document the position of the mechanical tubal occlusion contraceptive device 100.

The patient can then be discharged home, after being advised not to have any sexual activity for 4 weeks and protected sex after that for a total of 12 weeks.

Any symptoms should be recorded on a form that is provided and can also be entered electronically.

She should return in 3 months for a repeat HSG to confirm complete occlusion of both fallopian tube FT, due to dense scar formation. If both fallopian tubes FT are completely occluded, she can discontinue birth control pills and have unprotected sex. She should have follow-up checks for one year for symptoms or any pregnancy.

Patients should also be followed-up long term for 3 years for symptoms and any occurrence of pregnancies.

### Tubal Occlusion Contraceptive Appliance 200

Referring to FIGS. 25-27, after the baseline HSG is performed, the cervical OS can be dilated with serial dilators to about 5 mm. A 6 Fr JR 4 type guidewire delivery catheter GdC can be used to cannulate the right fallopian tube FT. A 0.018″ 80 cm guidewire G can be used to wire a first fallopian tube FT. The guidewire delivery catheter GdC is then exchanged out, leaving the guidewire G in place. A tubal occlusion contraceptive appliance 200 is loaded onto the guidewire G through the guideway(s) 218 and/or 228 in the contraceptive appliance 200. This allows the contraceptive appliance 200 to be advanced over the guidewire G in a mono rail fashion. The guidewire G is held firmly. The pusher delivery catheter PdC is advanced, with intermittent use of the fluoroscopy as desired. When the distal tip 210ₓ₂ of the contraceptive appliance 200 reaches the ostium of the fallopian tube FT, the freeze frame (road map) from the HSG should be reviewed. Using this as a guide the pusher delivery catheter PdC can be firmly advanced until the contraceptive appliance 200 advances into the fallopian tube FT and forward progress beyond the cornu is halted by the cap 220 of the contraceptive appliance 200. The contraceptive appliance 200 should be allowed to rest for a couple of minutes. Mild traction, will ensure that the contraceptive appliance 200 is locked in place. The guidewire G is then gently pulled out, while firmly holding the pusher delivery catheter PdC. Once the guidewire G is removed, the contraceptive appliance 200 can be deployed by firmly holding the pusher delivery catheter PdC in place while an assistant unscrews the lure lock screw cap over the hub H and unwinds the Nitinol tether T. The two ends of the tether T are separated, one of the wires is pulled until the entire wire comes out, and the pusher delivery catheter PdC is removed from the uterus.

The same procedure is repeated for the other fallopian tube FT. The JR4 guidewire delivery catheter GdC is re-introduced and the other fallopian tube FT is cannulated. It is then wired and the above steps are repeated in the other fallopian tube FT.

A final plain cine run will document the position of the two contraceptive appliance 200. The post procedure care is similar to that set forth previously.

We claim:

1. A mechanical tubal occlusion contraceptive device, comprising:
(a) an axially elongated scar-inducing occlusion element having first and second axial end portions terminating at first and second axial ends, respectively,
(b) an axially elongated leading anchor secured to and extending in a first axial direction from the first axial end portion of the occlusion element, the leading anchor including a plurality of radially extending barbs operable for resisting axial movement of the leading anchor relative to a fallopian tube into which the mechanical tubal occlusion contraceptive device has been implanted, and
(b) an axially elongated trailing anchor secured to and extending in a second axial direction opposite the first axial direction from the second axial end of the occlusion element, the trailing anchor including:
(ii) a plurality of radially extending barbs operable for resisting axial movement of the trailing anchor relative to a fallopian tube into which the mechanical tubal occlusion contraceptive device has been implanted, and
(iii) a head proximate a free axial end of the trailing anchor, configured and arranged for releasable engagement by a pusher delivery catheter for delivery of the mechanical tubal occlusion contraceptive device into a fallopian tube.

2. The mechanical tubal occlusion contraceptive device of claim 1, wherein the occlusion element is a flexible multifilament cord comprised of biocompatible polyester filaments.

3. The mechanical tubal occlusion contraceptive device of claim 1, wherein the leading and trailing anchors are comprised of a material selected from stainless steel and biocompatible polymers.

4. The mechanical tubal occlusion contraceptive device of claim 1, wherein:
(a) the leading anchor includes an axially extending first sleeve encasing the first axial end portion of the occlusion element,
(b) the trailing anchor includes an axially extending second sleeve encasing the second axial end portion of the occlusion element,
(c) the first and second sleeves are mechanically crimped onto the occlusion element, and
(d) a central axial portion of the occlusion element remains exposed.

5. The mechanical tubal occlusion contraceptive device of claim 4 wherein the exposed central axial portion of the occlusion element has an axial length of about 2 to 5 cm.

6. The mechanical tubal occlusion contraceptive device of claim 1, wherein the device has an axial length of about 4 to 7 cm and a radial cross-sectional area of about 1 to 3 mm².

7. The mechanical tubal occlusion contraceptive device of claim 1, wherein the leading anchor and trailing anchor are mirror images of one another.

8. The mechanical tubal occlusion contraceptive device of claim 1, wherein the barbs of both the leading anchor and the trailing anchor are axially spaced, radially projecting, conical discs.

9. The mechanical tubal occlusion contraceptive device of claim 4, wherein (i) the barbs of the leading anchor project radially from a shaft which projects in the first axial direction from the first sleeve, and (ii) the barbs of the trailing anchor project radially from a shaft which projects in the second axial direction from the second sleeve and is axially positioned between the second sleeve and the head.

10. The mechanical tubal occlusion contraceptive device of claim 1, wherein (i) at least one of the barbs on each of the leading and trailing anchors preferentially resists axial movement of the mechanical tubal occlusion contraceptive device in the first axial direction relative to a fallopian tube into which the mechanical tubal occlusion contraceptive device has been implanted, and (ii) at least one of the barbs on each of the leading and trailing anchors preferentially resists axial movement of the mechanical tubal occlusion contraceptive device in the second axial direction relative to a fallopian tube into which the mechanical tubal occlusion contraceptive device has been implanted.

11. The mechanical tubal occlusion contraceptive device of claim 1, wherein the head includes a radial through hole configured and arranged to accommodate passage of a releasable tether extending from a distal tip of a pusher delivery catheter for securing the tip of the catheter to the head.

12. A mechanical tubal occlusion contraceptive device, comprising:
(a) an axially elongated shaft having a proximal axial end spaced in a first axial direction from a distal tip, and defining a second axial direction opposite the first axial direction,
(b) a head projecting in the first axial direction from the proximal end of the shaft, configured and arranged for releasable engagement by a pusher delivery catheter for delivery of the mechanical tubal occlusion contraceptive device into a fallopian tube,
(c) a plurality of barbs extending radially from the shaft intermediate the cap and the distal tip, the barbs collectively resisting axial movement of the mechanical tubal occlusion contraceptive device in both the first and second axial directions relative to a fallopian tube into which the tubal occlusion contraceptive appliance has been implanted.

13. The mechanical tubal occlusion contraceptive device of claim 12, wherein (i) at least one of the barbs is configured and arranged to preferentially resist axial movement of the mechanical tubal occlusion contraceptive device in the first axial direction relative to a fallopian tube into which the mechanical tubal occlusion contraceptive device has been implanted, and (ii) at least one of the barbs is configured and arranged to preferentially resist axial movement of the mechanical tubal occlusion contraceptive device in the second axial direction relative to the fallopian tube into which the mechanical tubal occlusion contraceptive device has been implanted.

14. The mechanical tubal occlusion contraceptive device of claim 12, wherein the mechanical tubal occlusion contraceptive device is comprised of a material selected from stainless steel and a biocompatible polymer.

15. The mechanical tubal occlusion contraceptive device of claim 12, wherein the shaft has an axial length of about 1 to 4 cm and a radial cross-sectional area of about 0.8 to 7 mm².

16. The mechanical tubal occlusion contraceptive device of claim 13, wherein the barbs are axially spaced, radially projecting, conical discs.

17. A longitudinally elongated tubal occlusion contraceptive appliance, comprising:
(a) an axially elongated shaft having a proximal axial end spaced in a first axial direction from a distal axial end, and defining a second axial direction opposite the first axial direction,
(b) a guideway through the shaft proximate the distal axial end of the shaft operable for accommodating passage of a guidewire for guiding insertion of the appliance into a fallopian tube,
(c) a cap proximate the proximal axial end of the shaft sized, configured and arranged to inhibit axial travel of the cap into a fallopian tube, and
(d) a plurality of barbs extending radially from the shaft intermediate the distal axial end and the cap, the barbs configured and arranged to facilitate slide fit insertion of the shaft into a fallopian tube in the second axial direction while preferentially resisting axial movement of the shaft in the first axial direction out from the fallopian tube once inserted.

18. The tubal occlusion contraceptive appliance of claim 17, wherein the appliance is comprised of a material selected from stainless steel and biocompatible polymers.

19. The tubal occlusion contraceptive appliance of claim 17, wherein the shaft has an axial length of about 1 to 4 cm and a radial cross-sectional area of about 0.8 to 7 mm².

20. The tubal occlusion contraceptive appliance of claim 17, wherein the guideway angles inward in the second axial direction from a side of the shaft to the distal end of the shaft.

21. The tubal occlusion contraceptive appliance of claim 17, wherein the barbs are axially spaced, radially projecting, conical discs.

22. The tubal occlusion contraceptive appliance of claim 17, wherein the cap has a radial diameter greater than the radial diameter of the shaft.

23. The tubal occlusion contraceptive appliance of claim 17, wherein the cap has a radial diameter greater than the radial diameter of each barb.

24. The tubal occlusion contraceptive appliance of claim 17, wherein the cap includes a radial through hole configured and arranged to accommodate passage of a releasable tether extending from a distal tip of a pusher delivery catheter for securing the distal tip of the catheter to the cap.

25. A longitudinally elongated tubal occlusion contraceptive appliance, comprising:
(a) an axially elongated shaft having a proximal axial end spaced in a first axial direction from a distal tip, and defining a second axial direction opposite the first axial direction,
(b) a cap proximate the proximal end of the shaft sized, configured and arranged to inhibit axial travel of the cap into a fallopian tube,
(c) an axially extending proximal guideway through the cap radially spaced from the shaft operable for accommodating passage of a guidewire for guiding insertion of the appliance into the fallopian tube,
(d) an axially extending distal guideway through the shaft proximate the distal tip of the shaft operable for accommodating passage of the guidewire for guiding insertion of the appliance into a fallopian tube,
(e) a plurality of barbs extending radially from the shaft intermediate the distal tip of the shaft and the cap, the barbs configured and arranged to facilitate a second axial direction slide fit insertion of the shaft into a fallopian tube while preferentially resisting axial movement of the shaft in the first axial direction out from the fallopian tube once inserted.

26. The tubal occlusion contraceptive appliance of claim 25, wherein the appliance is comprised of a material selected from stainless steel and biocompatible polymers.

27. The tubal occlusion contraceptive appliance of claim 25, wherein the shaft has an axial length of about 1 to 4 cm and a radial cross-sectional area of about 0.8 to 7 mm².

28. The tubal occlusion contraceptive appliance of claim 25, wherein the distal guideway angles inward in the second axial direction from a side of the shaft to the distal tip of the shaft.

29. The tubal occlusion contraceptive appliance of claim 25, wherein the barbs are axially spaced, radially projecting, conical discs.

30. The tubal occlusion contraceptive appliance of claim 25, wherein the cap (i) has a radial diameter greater than the radial diameter of the shaft, and (ii) is configured and arranged for releasable threaded engagement by a pusher delivery catheter for delivery of the tubal occlusion contraceptive appliance into a fallopian tube.

31. The tubal occlusion contraceptive appliance of claim 25, wherein the cap has a radial diameter greater than the radial diameter of each barb.

32. A longitudinally elongated tubal occlusion contraceptive appliance, comprising:
(a) an axially elongated shaft having a proximal axial end spaced in a first axial direction from a distal tip, and defining a second axial direction opposite the first axial direction,
(b) a cap proximate the proximal end of the shaft sized, configured and arranged to inhibit travel of the cap into a fallopian tube,
(c) an axially extending proximal guideway through the cap radially spaced from the shaft operable for accommodating passage of a guidewire for guiding insertion of the appliance into a fallopian tube,
(d) an axially extending distal guideway through the shaft proximate the distal tip of the shaft operable for accommodating passage of the guidewire for guiding insertion of the appliance into the fallopian tube,
(e) screw threads extending radially around the shaft intermediate the distal tip of the shaft and the cap configured and arranged to (i) facilitate slide fit insertion of the shaft into a fallopian tube in the second axial direction with or without rotation of the appliance in a first direction while preferentially resisting sliding axial movement of the shaft in the first axial direction out from the fallopian tube once inserted, and (ii) effect withdrawal of the tubal occlusion contraceptive appliance from the fallopian tube upon rotation of the appliance in a second direction opposite the first direction.

33. The tubal occlusion contraceptive appliance of claim 32, wherein the cap is configured and arranged for releasable threaded engagement by a pusher delivery catheter for delivery of the appliance into a fallopian tube.

34. The tubal occlusion contraceptive appliance of claim 32, wherein the appliance is comprised of a material selected from stainless steel and a biocompatible polymer.

35. The tubal occlusion contraceptive appliance of claim 32, wherein the shaft has an axial length of about 1 to 4 cm and a radial cross-sectional area of about 0.8 to 7 mm².

36. The tubal occlusion contraceptive appliance of claim 32, wherein the distal guideway angles inward in the second axial direction from a side of the shaft to the first end of the shaft.

37. The tubal occlusion contraceptive appliance of claim 32, wherein the screw threads include a plurality of discrete, axially spaced, screw thread segments.

38. The tubal occlusion contraceptive appliance of claim 37, wherein each discrete screw thread segment has between 1 and 2 turns.

39. The tubal occlusion contraceptive appliance of claim 32, wherein the cap has a radial diameter greater than the radial diameter of the shaft.

40. The tubal occlusion contraceptive appliance of claim 32, wherein the cap has a radial diameter greater than the radial diameter of the screw threads.

41. The tubal occlusion contraceptive appliance of claim 32, wherein an exterior circumferential surface of the cap is knurled to facilitate gripping and rotation of the cap with a forceps to effect withdrawal of the tubal occlusion contraceptive appliance from a fallopian tube after insertion.

42. A longitudinally elongated tubal occlusion contraceptive appliance, comprising:
(a) an axially elongated shaft having a proximal axial end spaced in a first axial direction from a distal tip, and defining a second axial direction opposite the first axial direction,
(b) a cap proximate the proximal end of the shaft sized, configured and arranged to inhibit travel of the cap into a fallopian tube,
(c) an axially extending proximal guideway through the cap radially spaced from the shaft operable for accommodating passage of a guidewire for guiding insertion of the appliance into a fallopian tube,
(d) an axially extending distal guideway through the shaft proximate the distal tip of the shaft operable for accommodating passage of the guidewire for guiding insertion of the appliance into the fallopian tube,
(e) screw threads extending radially around the shaft for effecting axial insertion of the tubal occlusion contraceptive appliance into a fallopian tube upon rotation of the appliance in a first direction, and withdrawal of the tubal occlusion contraceptive appliance from the fallopian tube upon rotation of the appliance in a second direction opposite the first direction.

43. The tubal occlusion contraceptive appliance of claim 42, wherein the cap is configured and arranged for releasable threaded engagement by a pusher delivery catheter for delivery of the appliance into a fallopian tube.

44. The tubal occlusion contraceptive appliance of claim 42, wherein the appliance is comprised of a material selected from stainless steel and a biocompatible polymer.

45. The tubal occlusion contraceptive appliance of claim 42, wherein the shaft has an axial length of about 1 to 4 cm and a radial cross-sectional area of about 0.8 to 7 mm².

46. The tubal occlusion contraceptive appliance of claim 42, wherein the distal guideway angles inward in the second axial direction from a side of the shaft to the distal tip of the shaft.

47. The tubal occlusion contraceptive appliance of claim 42, wherein (i) the proximal guideway defines a first central axis, (ii) the distal guideway defines a second central axis, and (iii) the first central axis and the second central axis extend along a common line.

48. The tubal occlusion contraceptive appliance of claim 42, wherein the cap has a radial diameter greater than the radial diameter of the shaft.

49. The tubal occlusion contraceptive appliance of claim 42, wherein the cap has a radial diameter greater than the radial diameter of the screw threads.

50. The tubal occlusion contraceptive appliance of claim 42, wherein an exterior circumferential surface of the cap is knurled to facilitate gripping and rotation of the cap with a forceps to effect withdrawal of the tubal occlusion contraceptive appliance from a fallopian tube after insertion.

51. A method of implanting the mechanical tubal occlusion contraceptive device of claim 1, comprising the steps of:
(a) transvaginal introduction of a sheath having a lumen into a fallopian tube with an introduction end of the sheath exterior the vagina and a delivery end of the sheath within the fallopian tube,
(b) axially pushing the mechanical tubal occlusion contraceptive device along the lumen of the sheath with a delivery device from the introduction end to proximate the delivery end of the sheath, and
(c) withdrawing the delivery device and the sheath while leaving the mechanical tubal occlusion contraceptive device within the fallopian tube.

52. A method of implanting the mechanical tubal occlusion contraceptive device of claim 12, comprising the steps of:
(a) transvaginal introduction of a sheath having a lumen into a fallopian tube with an introduction end of the sheath exterior the vagina and a delivery end of the sheath within the fallopian tube,
(b) axially pushing the mechanical tubal occlusion contraceptive device along the lumen of the sheath with a delivery device from the introduction end to proximate the delivery end of the sheath, and
(c) withdrawing the delivery device and the sheath while leaving the mechanical tubal occlusion contraceptive device within the fallopian tube.

53. A method of implanting the tubal occlusion contraceptive appliance of claim 17, comprising the steps of:
(a) transvaginal introduction of a guidewire into a fallopian tube with a first end of the guidewire exterior the vagina and a second end of the guidewire within the fallopian tube,
(b) sliding the first end of the guidewire through the guideway in the tubal occlusion contraceptive appliance,
(c) axially pushing the tubal occlusion contraceptive appliance along the guidewire towards the second end of the guidewire with a delivery device until the shaft is positioned within the fallopian tube and the cap is seated over the opening from the uterus to the fallopian tube, and
(d) withdrawing the delivery device and the guidewire while leaving the tubal occlusion contraceptive appliance extending into the fallopian tube.

54. A method of implanting the tubal occlusion contraceptive appliance of claim 25, comprising the steps of:
(a) transvaginal introduction of a guidewire into a fallopian tube with a first end of the guidewire exterior the vagina and a second end of the guidewire within the fallopian tube,
(b) serially sliding the first end of the guidewire through the distal guideway and then through the proximal guideway in the tubal occlusion contraceptive appliance,
(c) axially pushing the tubal occlusion contraceptive appliance along the guidewire towards the second end of the guidewire with a delivery device until the shaft is positioned within the fallopian tube and the cap is seated over the opening from the uterus into the fallopian tube, and
(d) withdrawing the delivery device and the guidewire while leaving the tubal occlusion contraceptive appliance extending into the fallopian tube.

55. A method of implanting the tubal occlusion contraceptive appliance of claim 32, comprising the steps of:
(a) transvaginal introduction of a guidewire into a fallopian tube with a first end of the guidewire exterior the vagina and a second end of the guidewire within the fallopian tube,
(b) serially sliding the first end of the guidewire through the distal guideway and then through the proximal guideway in the tubal occlusion contraceptive appliance,
(c) axially pushing the tubal occlusion contraceptive appliance along the guidewire towards the second end of the guidewire with a delivery device until the shaft is positioned within the fallopian tube and the cap is seated over the opening from the uterus into the fallopian tube, and
(d) withdrawing the delivery device and the guidewire while leaving the tubal occlusion contraceptive appliance extending into the fallopian tube.

56. A method of implanting the tubal occlusion contraceptive appliance of claim 42, comprising the steps of:
(a) transvaginal introduction of a guidewire into a fallopian tube with a first end of the guidewire exterior the vagina and a second end of the guidewire within the fallopian tube,
(b) serially sliding the first end of the guidewire through the distal guideway and then through the proximal guideway in the tubal occlusion contraceptive appliance,
(c) axially pushing the tubal occlusion contraceptive appliance along the guidewire towards the second end of the guidewire with a delivery device until the screw threads on the shaft contact the fallopian tube,
(d) rotating the tubal occlusion contraceptive appliance in the first direction until the cap is seated over the opening from the uterus into the fallopian tube, and
(e) withdrawing the delivery device and the guidewire while leaving the tubal occlusion contraceptive appliance extending into the fallopian tube.

57. A method of removing an implanted tubal occlusion contraceptive appliance according to claim 32, comprising the steps of:
(a) rotating the cap about the axial axis of the implanted tubal occlusion contraceptive appliance whereby the screw threads on the implanted tubal occlusion contraceptive appliance effect axial travel of the implanted tubal occlusion contraceptive appliance completely out from the fallopian tube to create a withdrawn appliance, and then
(b) removing the withdrawn appliance completely from the body.

58. A method of removing an implanted tubal occlusion contraceptive appliance according to claim 32, comprising the steps of:
(a) gripping the cap with a forceps,
(b) rotating the cap with the forceps about the axial axis of the implanted tubal occlusion contraceptive appliance whereby the screw threads on the implanted tubal occlusion contraceptive appliance effect axial travel of the implanted tubal occlusion contraceptive appliance completely out from the fallopian tube to create a withdrawn appliance, and then
(c) removing the withdrawn appliance completely from the body.

59. A method of removing an implanted tubal occlusion contraceptive appliance according to claim 42, comprising the steps of:
(a) gripping the cap with a forceps,
(b) rotating the cap with the forceps about the axial axis of the implanted tubal occlusion contraceptive appliance whereby the screw threads on the implanted tubal occlusion contraceptive appliance effect axial travel of the implanted tubal occlusion contraceptive appliance completely out from the fallopian tube to create a withdrawn appliance, and then
(c) removing the withdrawn appliance completely from the body.
